(19) United States
(12) Patent Application Publication
(10) Pub. No.: US 2025/0170226 A1
(43) Pub. Date: May 29, 2025

(54) Methods Of Treating Skin Cancer With Histidine Ammonia-Lyase (HAL) Agonists

(71) Applicant: Regeneron Pharmaceuticals, Inc., Tarrytown, NY (US)

(72) Inventors: Manuel Allen Revez Ferreira, Tarrytown, NY (US); Joshua Backman, Tarrytown, NY (US); Alexander Li, Tarrytown, NY (US); Michael Kessler, Tarrytown, NY (US); Eric Jorgenson, Tarrytown, NY (US); Aris Baras, Tarrytown, NY (US); Goncalo Abecasis, Tarrytown, NY (US)

(21) Appl. No.: 18/962,276

(22) Filed: Nov. 27, 2024

Related U.S. Application Data
(63) Continuation of application No. 17/810,816, filed on Jul. 5, 2022, now Pat. No. 12,186,378.
(60) Provisional application No. 63/219,460, filed on Jul. 8, 2021.

(51) Int. Cl. A61K 38/51; A61K 31/198; A61P 35/00; C12Q 1/6869; C12Q 1/6886

(52) U.S. Cl. CPC A61K 38/51; A61K 31/198; A61P 35/00; C12Q 1/6886; C12Q 1/6869; C12Q 2600/156

(57) ABSTRACT

The present disclosure provides methods of treating subjects having skin cancer or at risk of developing skin cancer, and methods of identifying subjects having an increased risk of developing skin cancer.

### REFERENCE TO SEQUENCE LISTING

This application includes a Sequence Listing submitted electronically as a text file named 381204333SEQ, created on Nov. 21, 2024, with a size of 167,753 bytes. The Sequence Listing is incorporated herein by reference.

### FIELD

The present disclosure relates generally to the treatment of subjects having skin cancer or at risk of developing skin cancer with Histidine Ammonia-Lyase (HAL) agonists, and methods of identifying subjects having an increased risk of developing skin cancer.

### BACKGROUND

Skin cancer refers to all cancers that occur in the skin. These relatively common cancers are often mistaken by patients for non-malignant skin abnormalities, which can result in late detection that leads to difficulties in treating the disease and fatal outcomes. The most common of skin cancers is basal cell carcinoma (BCC), which accounts for about 80% of all skin cancers. Other types of skin cancers are squamous cell carcinoma (SCC), which accounts for approximately 16%, of all skin cancers, and melanoma, which accounts for about 4%. BCC and SCC are collectively referred to as non-melanoma skin cancer (NMSC). Melanoma occurs from melanocytes in the epidermis, many of which are metastatic cancers or carcinomas that lead to death. In 2000, 47,000 people were identified as having new melanomas, of which 7,700 were reported to have died (Greenlee et al., Cancer J. Clin., 2000, 50, 7-33. It is estimated that melanoma caused by ultraviolet rays is caused by intermittent exposure, such as intense tanning rather than chronic exposure to ultraviolet rays (Gilchrest et al., New Engl. J. Med., 1999, 340, 1341-1348). Another rare form of aggressive skin cancer is Merkel cell carcinoma (MCC), which is similar to melanoma.

The HAL gene encodes the enzyme histidine ammonia-lyase (or histidase) that converts histidine (an essential amino acid that is incorporated into fillaggrin, among other functions) into trans-urocanic acid, a major ultraviolet (UV)-absorbing chromophore that accumulates in the stratum corneum (Barresi et al., J. Invest. Dermatol., 2011, 131, 188-194). Inactivation of histidase is expected to decrease the ability of the outermost layer of the epidermis to block UV light.

### SUMMARY

The present disclosure provides methods of treating a subject having skin cancer or at risk of developing skin cancer, the methods comprising administering a HAL agonist to the subject.

The present disclosure also provides methods of treating a subject having non-melanoma skin cancer or preventing a subject from developing non-melanoma skin cancer, the methods comprising administering a HAL agonist to the subject.

The present disclosure also provides methods of treating a subject having basal cell carcinoma or preventing a subject from developing basal cell carcinoma, the methods comprising administering a HAL agonist to the subject.

The present disclosure also provides methods of treating a subject having squamous cell carcinoma or preventing a subject from developing squamous cell carcinoma, the methods comprising administering a HAL agonist to the subject.

The present disclosure also provides methods of treating a subject having melanoma or preventing a subject from developing melanoma, the methods comprising administering a HAL agonist to the subject.

The present disclosure also provides methods of treating a subject having Merkel cell carcinoma or preventing a subject from developing Merkel cell carcinoma, the methods comprising administering a HAL agonist to the subject.

The present disclosure also provides methods of treating a subject having dermatofibrosarcoma protuberans or preventing a subject from developing dermatofibrosarcoma protuberans, the methods comprising administering a HAL agonist to the subject.

The present disclosure also provides methods of treating a subject having sebaceous carcinoma or preventing a subject from developing sebaceous carcinoma, the methods comprising administering a HAL agonist to the subject.

The present disclosure also provides methods of treating a subject with a therapeutic agent that treats or prevents skin cancer, wherein the subject has skin cancer or is at risk for developing skin cancer, the methods comprising: determining whether the subject has a HAL variant nucleic acid molecule encoding a HAL predicted gain-of-function polypeptide by: obtaining or having obtained a biological sample from the subject; and performing or having performed a sequence analysis on the biological sample to determine if the subject has a genotype comprising the HAL variant nucleic acid molecule encoding the HAL predicted gain-of-function polypeptide; and administering or continuing to administer the therapeutic agent that treats or prevents skin cancer in a standard dosage amount to a subject that is HAL reference, and/or administering a HAL agonist to the subject; and administering or continuing to administer the therapeutic agent that treats or prevents skin cancer in an amount that is the same as or less than a standard dosage amount to a subject that is heterozygous for the HAL variant nucleic acid molecule, and/or administering a HAL agonist to the subject; wherein the presence of a genotype having the HAL variant nucleic acid molecule encoding the HAL predicted gain-of-function polypeptide indicates the subject has a decreased risk of developing skin cancer.

The present disclosure also provides methods of identifying a subject having an increased risk of developing skin cancer, the methods comprising: determining or having determined the presence or absence of a HAL variant nucleic acid molecule encoding a HAL predicted gain-of-function polypeptide in a biological sample obtained from the subject; wherein: when the subject is HAL reference, then the subject has an increased risk of developing skin cancer; and when the subject is heterozygous or homozygous for a HAL variant nucleic acid molecule encoding the HAL predicted gain-of-function polypeptide, then the subject has a decreased risk of developing skin cancer.

The present disclosure also provides therapeutic agents that treat or prevent skin cancer for use in the treatment or prevention of skin cancer in a subject identified as having a genomic nucleic acid molecule encoding a HAL predicted gain-of-function polypeptide, or the complement thereof, wherein the genomic nucleic acid molecule has a nucleotide sequence comprising: i) an adenine at a position corresponding to position 11,352 according to SEQ ID NO:2, or the complement thereof; or ii) a guanine at a position corresponding to position 14,441 according to SEQ ID NO:3, or the complement thereof.

The present disclosure also provides HAL agonists for use in the treatment or prevention of skin cancer in a subject that: a) is reference for a HAL genomic nucleic acid molecule, a HAL mRNA molecule, or a HAL cDNA molecule; or b) is heterozygous for a genomic nucleic acid molecule encoding a HAL predicted gain-of-function polypeptide, or the complement thereof, wherein the genomic nucleic acid molecule has a nucleotide sequence comprising: i) an adenine at a position corresponding to position 11,352 according to SEQ ID NO:2, or the complement thereof; or ii) a guanine at a position corresponding to position 14,441 according to SEQ ID NO:3, or the complement thereof.

### BRIEF DESCRIPTION OF THE DRAWINGS

The accompanying figures, which are incorporated in and constitute a part of this specification, illustrate several features of the present disclosure.

FIG. 1 shows an association between a non-coding variant in HAL and gene expression in skin tissue (sun exposed—lower leg) from GTEx.

FIG. 2 shows an association between non-coding variants in HAL and vitamin D levels and skin cancer-related traits.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is not intended that any method or aspect set forth herein be construed as requiring that its steps be performed in a specific order. Accordingly, where a method claim does not specifically state in the claims or descriptions that the steps are to be limited to a specific order, it is not intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or other tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

A burden of common putative gain-of-function (GOF) in the HAL gene associated with a decreased risk of developing skin cancer in humans has been identified in accordance with the present disclosure. For example, a genetic alteration that changes the guanine at position 11,352 in the HAL reference genomic nucleic acid molecule (see, SEQ ID NO:1) to an adenine or changes the adenine at position 14,441 in the HAL reference genomic nucleic acid molecule to a guanine, has been observed to indicate that the subject having such an alteration may have a decreased risk of developing skin cancer. Altogether, the genetic analyses described herein surprisingly indicate that the HAL gene and, in particular, pGOFs in the HAL gene, associates with a decreased risk of developing skin cancer. Therefore, subjects that are HAL reference that have an increased risk of developing skin cancer, such as non-melanoma skin cancer, basal cell carcinoma, squamous cell carcinoma, melanoma, Merkel cell carcinoma, dermatofibrosarcoma protuberans, or sebaceous carcinoma, may be treated such that the skin cancer is prevented, the symptoms thereof are reduced, and/or development of symptoms is repressed. Accordingly, the present disclosure provides methods of leveraging the identification of such variants in subjects to identify or stratify risk in such subjects of developing skin cancer, such as non-melanoma skin cancer, basal cell carcinoma, squamous cell carcinoma, melanoma, Merkel cell carcinoma, dermatofibrosarcoma protuberans, or sebaceous carcinoma, or to diagnose subjects as having an increased risk of developing skin cancer, such as non-melanoma skin cancer, basal cell carcinoma, squamous cell carcinoma, melanoma, Merkel cell carcinoma, dermatofibrosarcoma protuberans, or sebaceous carcinoma, such that subjects at risk or subjects with active disease may be treated accordingly.

It has been further observed in accordance with the present disclosure that HAL variant nucleic acid molecules encoding a HAL predicted gain-of-function polypeptide (whether these variations are homozygous or heterozygous in a particular subject) associate with a decreased risk of developing skin cancer. Moreover, the identification by the present disclosure of the association between additional variants and gene burden masks indicates that HAL may be responsible for a protective effect in skin cancer.

For purposes of the present disclosure, any particular subject can be categorized as having one of three HAL genotypes: i) HAL reference; ii) heterozygous for a HAL variant nucleic acid molecule encoding a HAL predicted gain-of-function polypeptide; or iii) homozygous for a HAL variant nucleic acid molecule encoding a HAL predicted gain-of-function polypeptide. A subject is HAL reference when the subject does not have a copy of a HAL variant nucleic acid molecule encoding a HAL predicted gain-of-function polypeptide. A subject is heterozygous for a HAL variant nucleic acid molecule encoding a HAL predicted gain-of-function polypeptide when the subject has a single copy of a HAL variant nucleic acid molecule. As used herein, a HAL variant nucleic acid molecule is any HAL nucleic acid molecule (such as, a genomic nucleic acid molecule, an mRNA molecule, or a cDNA molecule) encoding a HAL polypeptide having a partial gain-of-function, a complete gain-of-function, a predicted partial gain-of-function, or a predicted complete gain-of-function. A subject who has a HAL variant nucleic acid molecule encoding a HAL predicted gain-of-function polypeptide having a partial gain-of-function (or predicted partial gain-of-function) is hypomorphic for HAL. A subject is homozygous for a HAL variant nucleic acid molecule encoding a HAL predicted gain-of-function polypeptide when the subject has two copies of a HAL variant nucleic acid molecule encoding a HAL predicted gain-of-function polypeptide.

For subjects that are genotyped or determined to be HAL reference, such subjects have an increased risk of developing skin cancer, such as non-melanoma skin cancer, basal cell carcinoma, squamous cell carcinoma, melanoma, Merkel cell carcinoma, dermatofibrosarcoma protuberans, and/or sebaceous carcinoma. For subjects that are genotyped or determined to be either HAL reference or heterozygous for a HAL variant nucleic acid molecule encoding a HAL predicted gain-of-function polypeptide, such subjects can be treated with a HAL agonist.

In any of the embodiments described throughout the present disclosure, the HAL variant nucleic acid molecule can be any HAL nucleic acid molecule (such as, for example, genomic nucleic acid molecule, mRNA molecule, or cDNA molecule) encoding a HAL polypeptide having a partial gain-of-function, a complete gain-of-function, a predicted partial gain-of-function, or a predicted complete gain-of-function.

In any of the embodiments described throughout the present disclosure, the HAL predicted gain-of-function polypeptide can be any HAL polypeptide having a partial gain-of-function, a complete gain-of-function, a predicted partial gain-of-function, or a predicted complete gain-of-function.

Any one or more (i.e., any combination) of the HAL variant nucleic acid molecules encoding a HAL predicted gain-of-function polypeptide can be used within any of the methods described herein to determine whether a subject has an increased risk of developing skin cancer. The combinations of particular variants can form a mask used for statistical analysis of the particular correlation of HAL and decreased risk of developing skin cancer.

In any of the embodiments described throughout the present disclosure, the skin cancer is non-melanoma skin cancer, basal cell carcinoma, squamous cell carcinoma, melanoma, Merkel cell carcinoma, dermatofibrosarcoma protuberans, or sebaceous carcinoma. In any of the embodiments described throughout the present disclosure, the skin cancer is non-melanoma skin cancer. In any of the embodiments described throughout the present disclosure, the skin cancer is basal cell carcinoma. In any of the embodiments described throughout the present disclosure, the skin cancer is squamous cell carcinoma. In any of the embodiments described throughout the present disclosure, the skin cancer is melanoma. In any of the embodiments described throughout the present disclosure, the skin cancer is Merkel cell carcinoma. In any of the embodiments described throughout the present disclosure, the skin cancer is dermatofibrosarcoma protuberans. In any of the embodiments described throughout the present disclosure, the skin cancer is sebaceous carcinoma.

Symptoms of basal cell carcinoma include, but are not limited to, a raised, smooth, pearly bump on the sun-exposed skin of an individual's head, neck or shoulders. Often small blood vessels can be seen within the tumor. Crusting of the tumor, as well as bleeding can occur. Individuals sometimes mistake basal cell carcinoma as a sore that will not heal. Basal cell carcinoma is the least deadly form of skin cancer and often times with proper treatment can be completely eliminated.

Symptoms of squamous cell carcinoma include, but are not limited to a red, scaling, thickened patch on the sun exposed skin of an individual. Some forms of squamous cell carcinoma appear as firm hard nodules and as dome shapes. Breaks and bleeding of the nodules may occur. If left untreated, the squamous cell carcinoma could develop into a large mass. Squamous cell carcinoma is the second most common form of skin cancer.

Symptoms of melanoma include, but are not limited to, shades or brown to black lesions. There are also some melanomas which appear pink, red or flesh color, these are called amelanotic melanomas. The amelanotic melanomas are a more aggressive form of melanoma. Some of the warning signs of malignant melanoma could include changes in size, shape, color, elevation of a mole, the development of a new mole in the transitional period from puberty to adulthood, itching, ulceration or bleeding. Melanoma is the most deadly form of skin cancer.

Symptoms of Merkel cell carcinoma include, but are not limited to rapid growing, non-tender flesh colored to red/violet bumps that are usually not painful or itchy. These bumps appear on the highly sun exposed skin of the head, neck and arms. Individuals often mistake Merkel cell carcinoma for a cyst or other type of cancer.

Symptoms of dermatofibrosarcoma protuberans include, but are not limited to small, slightly-raised, red or purple patch of skin 1 to 5 centimeters wide that can become a raised nodule and in some cases may cause redness, open up or bleed.

Symptoms of sebaceous carcinoma include, but are not limited to slow-growing sometimes yellow painless lump at an eyelid. The bump may bleed or ooze and may also have a thickening or yellow or reddish crust, where the eyelid meets the lash.

The present disclosure provides methods of treating a subject having skin cancer or at risk of developing skin cancer, the methods comprising administering a HAL agonist to the subject.

The present disclosure also provides methods of treating a subject having non-melanoma skin cancer or at risk of developing non-melanoma skin cancer, the methods comprising administering a HAL agonist to the subject.

The present disclosure also provides methods of treating a subject having basal cell carcinoma or at risk of developing basal cell carcinoma, the methods comprising administering a HAL agonist to the subject.

The present disclosure also provides methods of treating a subject having squamous cell carcinoma or at risk of developing squamous cell carcinoma, the methods comprising administering a HAL agonist to the subject.

The present disclosure also provides methods of treating a subject having melanoma or at risk of developing melanoma, the methods comprising administering a HAL agonist to the subject.

The present disclosure also provides methods of treating a subject having Merkel cell carcinoma or at risk of developing Merkel cell carcinoma, the methods comprising administering a HAL agonist to the subject.

The present disclosure also provides methods of treating a subject having dermatofibrosarcoma protuberans or at risk of developing dermatofibrosarcoma protuberans, the methods comprising administering a HAL agonist to the subject.

The present disclosure also provides methods of treating a subject having sebaceous carcinoma or at risk of developing sebaceous carcinoma, the methods comprising administering a HAL agonist to the subject.

In some embodiments, the HAL agonist is HAL protein, or a functionally active fragment thereof, or thyroid hormone (T₃).

In some embodiments, the methods of treatment further comprise detecting the presence or absence of a HAL variant nucleic acid molecule encoding a HAL predicted gain-of-function polypeptide in a biological sample obtained from the subject. As used throughout the present disclosure, “a HAL variant nucleic acid molecule” is any HAL nucleic acid molecule (such as, for example, genomic nucleic acid molecule, mRNA molecule, or cDNA molecule) encoding a HAL polypeptide having a partial gain-of-function, a complete gain-of-function, a predicted partial gain-of-function, or a predicted complete gain-of-function.

The present disclosure also provides methods of treating a subject with a therapeutic agent that treats or prevents skin cancer. In some embodiments, the subject has skin cancer. In some embodiments, the subject is at risk of developing skin cancer. In some embodiments, the methods comprise determining whether the subject has a HAL variant nucleic acid molecule encoding a HAL predicted gain-of-function polypeptide by obtaining or having obtained a biological sample obtained from the subject, and performing or having performed a sequence analysis on the biological sample to determine if the subject has a genotype comprising the HAL variant nucleic acid molecule. When the subject is HAL reference, the therapeutic agent that treats or prevents skin cancer is administered or continued to be administered to the subject in a standard dosage amount, and/or a HAL agonist is administered to the subject. When the subject is heterozygous for a HAL variant, the therapeutic agent that treats or prevents skin cancer is administered or continued to be administered to the subject in an amount that is the same as or less than a standard dosage amount, and/or a HAL agonist is administered to the subject. The presence of a genotype having the HAL variant nucleic acid molecule encoding the HAL predicted gain-of-function polypeptide indicates the subject has a decreased risk of developing skin cancer. In some embodiments, the subject is HAL reference. In some embodiments, the subject is heterozygous for the HAL variant nucleic acid molecule encoding a HAL predicted gain-of-function polypeptide.

For subjects that are genotyped or determined to be either HAL reference or heterozygous for a HAL variant nucleic acid molecule encoding a HAL predicted gain-of-function polypeptide, such subjects can be treated with a HAL agonist, as described herein.

Detecting the presence or absence of a HAL variant nucleic acid molecule encoding a HAL predicted gain-of-function polypeptide in a biological sample obtained from a subject and/or determining whether a subject has a HAL variant nucleic acid molecule encoding a HAL predicted gain-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the HAL variant nucleic acid molecule encoding a HAL predicted gain-of-function polypeptide can be present within a cell obtained from the subject.

In some embodiments, when the subject is HAL reference, the subject is also administered a therapeutic agent that treats or prevents skin cancer in a standard dosage amount. In some embodiments, when the subject is heterozygous for a HAL variant nucleic acid molecule encoding a HAL predicted gain-of-function polypeptide, the subject is administered a therapeutic agent that treats or prevents skin cancer in a dosage amount that is the same as or less than a standard dosage amount.

In some embodiments, the treatment methods further comprise detecting the presence or absence of a HAL predicted gain-of-function polypeptide in a biological sample obtained from the subject. In some embodiments, when the subject does not have a HAL predicted gain-of-function polypeptide, the subject is administered a therapeutic agent that treats or prevents skin cancer in a standard dosage amount. In some embodiments, when the subject has a HAL predicted gain-of-function polypeptide, the subject is administered a therapeutic agent that treats or prevents skin cancer in a dosage amount that is the same as or less than a standard dosage amount.

The present disclosure also provides methods of treating a subject with a therapeutic agent that treats or prevents skin cancer. In some embodiments, the subject has skin cancer. In some embodiments, the subject is at risk of developing skin cancer. In some embodiments, the methods comprise determining whether the subject has a HAL predicted gain-of-function polypeptide by obtaining or having obtained a biological sample from the subject, and performing or having performed an assay on the biological sample to determine if the subject has a HAL predicted gain-of-function polypeptide. When the subject does not have a HAL predicted gain-of-function polypeptide, the therapeutic agent that treats or prevents skin cancer is administered or continued to be administered to the subject in a standard dosage amount, and/or a HAL agonist is administered to the subject. When the subject has a HAL predicted gain-of-function polypeptide, the therapeutic agent that treats or prevents skin cancer is administered or continued to be administered to the subject in an amount that is the same as or less than a standard dosage amount, and/or a HAL agonist is administered to the subject. The presence of a HAL predicted gain-of-function polypeptide indicates the subject has a decreased risk of developing skin cancer. In some embodiments, the subject has a HAL predicted gain-of-function polypeptide. In some embodiments, the subject does not have a HAL predicted gain-of-function polypeptide.

Detecting the presence or absence of a HAL predicted gain-of-function polypeptide in a biological sample obtained from a subject and/or determining whether a subject has a HAL predicted gain-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the HAL predicted gain-of-function polypeptide can be present within a cell obtained from the subject.

Examples of therapeutic agents that treat or prevent skin cancer include, but are not limited to: thiazide diuretics (such as, chlorthalidone, chlorothiazide, hydrochlorothiazide, indapamide, or metolazone); potassium-sparing diuretics (such as, amiloride, spironolactone, or triamterene); loop diuretics (such as, bumetanide, furosemide, or torsemide); beta blockers (such as, acebutolol, atenolol, betaxolol, bisoprolol, bisoprolol/hydrochlorothiazide, metoprolol tartrate, metoprolol succinate, nadolol, pindolol, propranolol, solotol, or timolol); angiotensin converting enzyme (ACE) agonists (such as, benazepril, captopril, enalapril, fosinopril, lisinopril, moexipril, perindopril, quinapril, ramipril, or trandolapril); angiotensin II receptor blockers (ARBs) (such as, candesartan, eprosartan, irbesartan, losartan, telmisartan, or valsartan); calcium channel blockers (such as, amlodipine, diltiazem, felodipine, isradipine, nicardipine, nifedipine, nisoldipine, or verapamil); alpha-blockers (such as, doxazosin, prazosin, or terazosin); alpha-beta-blockers (such as carvedilol or labetalol); central agonists (such as, methyldopa, clonidine, or guanfacine); vasodilators (such as, hydralazine or minoxidil); aldosterone receptor antagonists (such as, eplerenone or spironolactone), and renin agonists (such as aliskiren).

In some embodiments, the therapeutic agent that treats or prevents skin cancer is a thiazide diuretic, a potassium-sparing diuretic, a loop diuretic, a beta blocker, an ACE inhibitor, an ARB, a calcium channel blocker, an alpha-blocker, an alpha-beta-blocker, a central agonist, a vasodilator, an aldosterone receptor antagonist, or a renin inhibitor. In some embodiments, the thiazide diuretic is chlorthalidone, chlorothiazide, hydrochlorothiazide, indapamide, or metolazone. In some embodiments, the potassium-sparing diuretic is amiloride, spironolactone, or triamterene. In some embodiments, the loop diuretic is bumetanide, furosemide, ortorsemide. In some embodiments, the beta blocker is acebutolol, atenolol, betaxolol, bisoprolol, bisoprolol/hydrochlorothiazide, metoprolol tartrate, metoprolol succinate, nadolol, pindolol, propranolol, solotol, or timolol). In some embodiments, the ACE inhibitor is benazepril, captopril, enalapril, fosinopril, lisinopril, moexipril, perindopril, quinapril, ramipril, or trandolapril. In some embodiments, the ARB is candesartan, eprosartan, irbesartan, losartan, telmisartan, or valsartan. In some embodiments, the calcium channel blocker is amlodipine, diltiazem, felodipine, isradipine, nicardipine, nifedipine, nisoldipine, orverapamil. In some embodiments, the alpha-blocker is doxazosin, prazosin, or terazosin. In some embodiments, the alpha-beta-blocker is carvedilol or labetalol. In some embodiments, the central agonist is methyldopa, clonidine, or guanfacine). In some embodiments, the vasodilator is hydralazine or minoxidil. In some embodiments, the aldosterone receptor antagonist is eplerenone or spironolactone. In some embodiments, the renin inhibitor is aliskiren.

In some embodiments, the dose of the therapeutic agents that treat or prevent skin cancer can be reduced by about 10%, by about 20%, by about 30%, by about 40%, by about 50%, by about 60%, by about 70%, by about 80%, or by about 90% for subjects that are heterozygous for a HAL variant nucleic acid molecule encoding a HAL predicted gain-of-function polypeptide (i.e., less than the standard dosage amount) compared to subjects that are HAL reference (who may receive a standard dosage amount). In some embodiments, the dose of the therapeutic agents that treat or prevent skin cancer can be reduced by about 10%, by about 20%, by about 30%, by about 40%, or by about 50%. In addition, the dose of therapeutic agents that treat or prevent skin cancer in subjects that are heterozygous for a HAL variant nucleic acid molecule encoding a HAL predicted gain-of-function polypeptide can be administered less frequently compared to subjects that are HAL reference.

Administration of the therapeutic agents that treat or prevent skin cancer and/or HAL agonists can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more. In addition, the therapeutic agents that treat or prevent skin cancer and/or HAL agonists can be administered sequentially or at the same time. In addition, the therapeutic agents that treat or prevent skin cancer and/or HAL agonists can be administered in separate compositions or can be administered together in the same composition.

Administration of the therapeutic agents that treat or prevent skin cancer and/or HAL agonists can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in skin cancer, a decrease/reduction in the severity of skin cancer (such as, for example, a reduction or inhibition of development of skin cancer), a decrease/reduction in symptoms and skin cancer-related effects, delaying the onset of symptoms and skin cancer-related effects, reducing the severity of symptoms of skin cancer-related effects, reducing the severity of an acute episode, reducing the number of symptoms and skin cancer-related effects, reducing the latency of symptoms and skin cancer-related effects, an amelioration of symptoms and skin cancer-related effects, reducing secondary symptoms, reducing secondary infections, preventing relapse to skin cancer, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of skin cancer development/progression (such as, for example, a complete or partial avoidance/inhibition or a delay), and an increased survival time of the affected host animal, following administration of a therapeutic protocol. Treatment of skin cancer encompasses the treatment of subjects already diagnosed as having any form of skin cancer at any clinical stage or manifestation, the delay of the onset or evolution or aggravation or deterioration of the symptoms or signs of skin cancer, and/or preventing and/or reducing the severity of skin cancer.

The present disclosure also provides methods of identifying a subject having an increased risk of developing skin cancer. In some embodiments, the methods comprise determining or having determined the presence or absence of a HAL variant nucleic acid molecule (such as a genomic nucleic acid molecule, mRNA molecule, and/or cDNA molecule) encoding a HAL predicted gain-of-function polypeptide in a biological sample obtained from the subject. When the subject lacks a HAL variant nucleic acid molecule encoding a HAL predicted gain-of-function polypeptide (i.e., the subject is genotypically categorized as HAL reference), then the subject has an increased risk of developing skin cancer. When the subject has a HAL variant nucleic acid molecule encoding a HAL predicted gain-of-function polypeptide (i.e., the subject is heterozygous or homozygous for a HAL variant nucleic acid molecule), then the subject has a decreased risk of developing skin cancer compared to a subject that is HAL reference.

Having a single copy of a HAL variant nucleic acid molecule encoding a HAL predicted gain-of-function polypeptide is more protective of a subject from developing skin cancer than having no copies of a HAL variant nucleic acid molecule encoding a HAL predicted gain-of-function polypeptide. Without intending to be limited to any particular theory or mechanism of action, it is believed that a single copy of a HAL variant nucleic acid molecule (i.e., heterozygous for a HAL variant nucleic acid molecule) is protective of a subject from developing skin cancer, and it is also believed that having two copies of a HAL variant nucleic acid molecule encoding a HAL predicted gain-of-function polypeptide (i.e., homozygous for a HAL variant nucleic acid molecule) may be more protective of a subject from developing skin cancer, relative to a subject with a single copy. Thus, in some embodiments, a single copy of a HAL variant nucleic acid molecule encoding a HAL predicted gain-of-function polypeptide may not be completely protective, but instead, may be partially or incompletely protective of a subject from developing skin cancer. While not desiring to be bound by any particular theory, there may be additional factors or molecules involved in the development of skin cancer that are still present in a subject having a single copy of a HAL variant nucleic acid molecule encoding a HAL predicted gain-of-function polypeptide, thus resulting in less than complete protection from the development of skin cancer.

Detecting the presence or absence of a HAL variant nucleic acid molecule encoding a HAL predicted gain-of-function polypeptide in a biological sample obtained from a subject and/or determining whether a subject has a HAL variant nucleic acid molecule encoding a HAL predicted gain-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the HAL variant nucleic acid molecule encoding a HAL predicted gain-of-function polypeptide can be present within a cell obtained from the subject.

In some embodiments, when a subject is identified as having an increased risk of developing skin cancer, the subject is further treated with a therapeutic agent that treats or prevents skin cancer and/or a HAL agonist, as described herein. For example, when the subject is HAL reference, and therefore has an increased risk for developing skin cancer, the subject is administered a HAL agonist. In some embodiments, such a subject is also administered a therapeutic agent that treats or prevents skin cancer. In some embodiments, when the subject is heterozygous for a HAL variant nucleic acid molecule encoding a HAL predicted gain-of-function polypeptide, the subject is administered the therapeutic agent that treats or prevents skin cancer in a dosage amount that is the same as or less than a standard dosage amount, and/or is administered a HAL agonist. In some embodiments, the subject is HAL reference. In some embodiments, the subject is heterozygous for a HAL variant nucleic acid molecule encoding a HAL predicted gain-of-function polypeptide.

In some embodiments, any of the methods described herein can further comprise determining the subject's aggregate burden of having a HAL variant nucleic acid molecule encoding a HAL predicted gain-of-function polypeptide, and/or a HAL predicted gain-of-function variant polypeptide associated with a decreased risk of developing skin cancer. The aggregate burden is the aggregate of all variants in the HAL gene, which can be carried out in an association analysis with skin cancer. In some embodiments, the subject is homozygous for one or more HAL variant nucleic acid molecules encoding a HAL predicted gain-of-function polypeptide associated with a decreased risk of developing skin cancer. In some embodiments, the subject is heterozygous for one or more HAL variant nucleic acid molecules encoding a HAL predicted gain-of-function polypeptide associated with a decreased risk of developing skin cancer. The result of the association analysis suggests that HAL variant nucleic acid molecules encoding a HAL predicted gain-of-function polypeptide are associated with decreased risk of developing skin cancer. When the subject has a lower aggregate burden, the subject is at a higher risk of developing skin cancer and the subject is administered or continued to be administered the therapeutic agent that treats or prevents skin cancer in a standard dosage amount, and/or a HAL agonist. When the subject has a greater aggregate burden, the subject is at a lower risk of developing skin cancer and the subject is administered or continued to be administered the therapeutic agent that treats or prevents skin cancer in an amount that is the same as or less than the standard dosage amount. The greater the aggregate burden, the lower the risk of developing skin cancer.

HAL variants that can be used in the aggregate burden analysis include any one or more, or any combination, of the following Table 1:

TABLE 1

| Variant | rsID |
|---|---|
| 12:95980972:G:A | rs2270318 |
| 12:95981904:T:C | rs10859995 |
| 12:95984993:C:T | rs3819817 |
| 12:95986028:G:A | rs3213737 |

In some embodiments, the subject's aggregate burden of having any one or more HAL variant nucleic acid molecules encoding a HAL predicted gain-of-function polypeptide represents a weighted aggregate of a plurality of any of the HAL variant nucleic acid molecules encoding a HAL predicted gain-of-function polypeptide. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the HAL gene where the genetic burden is the number of alleles multiplied by the association estimate with skin cancer or related outcome for each allele (e.g., a weighted polygenic burden score). This can include any genetic variants, regardless of their genomic annotation, in proximity to the HAL gene (up to 10 Mb around the gene) that show a non-zero association with skin cancer-related traits in a genetic association analysis. In some embodiments, when the subject has an aggregate burden above a desired threshold score, the subject has a decreased risk of developing skin cancer. In some embodiments, when the subject has an aggregate burden below a desired threshold score, the subject has an increased risk of developing skin cancer.

In some embodiments, the aggregate burden may be divided into quintiles, e.g., top quintile, intermediate quintile, and bottom quintile, wherein the top quintile of aggregate burden corresponds to the lowest risk group and the bottom quintile of aggregate burden corresponds to the highest risk group. In some embodiments, a subject having a greater aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with skin cancer in the top 10%, top 20%, top 30%, top 40%, or top 50% of p-value range for the association. In some embodiments, each of the identified genetic variants comprise the genetic variants having association with skin cancer with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with skin cancer with p-value of less than 5×10⁻⁸. In some embodiments, the identified genetic variants comprise genetic variants having association with skin cancer in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having aggregate burdens in the bottom decile, quintile, or tertile in a reference population. The threshold of the aggregate burden is determined on the basis of the nature of the intended practical application and the risk difference that would be considered meaningful for that practical application.

In some embodiments, when a subject is identified as having an increased risk of developing skin cancer, the subject is further administered a therapeutic agent that treats or prevents skin cancer, and/or a HAL agonist, as described herein. For example, when the subject is HAL reference, and therefore has an increased risk of developing skin cancer, the subject is administered a HAL agonist. In some embodiments, such a subject is also administered a therapeutic agent that treats or prevents skin cancer. In some embodiments, when the subject is heterozygous for a HAL variant nucleic acid molecule encoding a HAL predicted gain-of-function polypeptide, the subject is administered the therapeutic agent that treats or prevents skin cancer in a dosage amount that is the same as or less than a standard dosage amount, and/or is administered a HAL agonist. In some embodiments, the subject is HAL reference. In some embodiments, the subject is heterozygous for a HAL variant nucleic acid molecule encoding a HAL predicted gain-of-function polypeptide. Furthermore, when the subject has a lower aggregate burden for having a HAL variant nucleic acid molecule encoding a HAL predicted gain-of-function polypeptide, and therefore has an increased risk of developing skin cancer, the subject is administered a therapeutic agent that treats or prevents skin cancer. In some embodiments, when the subject has a lower aggregate burden for having a HAL variant nucleic acid molecule encoding a HAL predicted gain-of-function polypeptide, the subject is administered the therapeutic agent that treats or prevents skin cancer in a dosage amount that is the same as or greater than the standard dosage amount administered to a subject who has a greater aggregate burden for having a HAL variant nucleic acid molecule encoding a HAL predicted gain-of-function polypeptide.

The present disclosure also provides methods of detecting the presence or absence of a HAL variant genomic nucleic acid molecule encoding a HAL predicted gain-of-function polypeptide in a biological sample obtained from a subject. It is understood that gene sequences within a population and mRNA molecules encoded by such genes can vary due to polymorphisms such as single nucleotide polymorphisms (SNPs). The sequences provided herein for the HAL variant genomic nucleic acid molecules are only exemplary sequences. Other sequences for the HAL variant genomic nucleic acid molecules are also possible.

The biological sample can be derived from any cell, tissue, or biological fluid from the subject. The biological sample may comprise any clinically relevant tissue such as, for example, a bone marrow sample, a tumor biopsy, a fine needle aspirate, or a sample of bodily fluid, such as blood, gingival crevicular fluid, plasma, serum, lymph, ascitic fluid, cystic fluid, or urine. In some embodiments, the biological sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any HAL variant nucleic acid molecule, preliminary processing designed to isolate or enrich the biological sample for the HAL variant nucleic acid molecule can be employed. A variety of techniques may be used for this purpose. When detecting the level of any HAL variant mRNA molecule, different techniques can be used enrich the biological sample with mRNA molecules. Various methods to detect the presence or level of an mRNA molecule or the presence of a particular variant genomic DNA locus can be used.

The present disclosure also provides methods of detecting a HAL variant nucleic acid molecule, or the complement thereof, encoding a HAL predicted gain-of-function polypeptide in a subject. The methods comprise assaying a biological sample obtained from the subject to determine whether a nucleic acid molecule in the biological sample is a HAL variant nucleic acid molecule encoding a HAL predicted gain-of-function polypeptide.

In some embodiments, the HAL variant nucleic acid molecule encoding the HAL predicted gain-of-function polypeptide, or the complement thereof, is a genomic nucleic acid molecule having a nucleotide sequence comprising: an adenine at a position corresponding to position 11,352 according to SEQ ID NO:2, or the complement thereof; or a guanine at a position corresponding to position 14,441 according to SEQ ID NO:3, or the complement thereof. In some embodiments, the HAL variant genomic nucleic acid molecule encoding the HAL predicted gain-of-function polypeptide, or the complement thereof, has a nucleotide sequence comprising an adenine at a position corresponding to position 11,352 according to SEQ ID NO:2, or the complement thereof. In some embodiments, the HAL variant genomic nucleic acid molecule encoding the HAL predicted gain-of-function polypeptide, or the complement thereof, has a nucleotide sequence comprising a guanine at a position corresponding to position 14,441 according to SEQ ID NO:3, or the complement thereof.

In some embodiments, the biological sample comprises a cell or cell lysate. Such methods can further comprise, for example, obtaining a biological sample from the subject comprising a HAL genomic nucleic acid molecule. Such assays can comprise, for example determining the identity of these positions of the particular HAL nucleic acid molecule. In some embodiments, the method is an in vitro method.

In some embodiments, the assay comprises sequencing at least a portion of the nucleotide sequence of the HAL nucleic acid molecule, or the complement thereof, in the biological sample. In some embodiments, the assay comprises sequencing at least a portion of the nucleotide sequence of the HAL genomic nucleic acid molecule in the biological sample, wherein the sequenced portion comprises: a position corresponding to position 11,352 according to SEQ ID NO:2, or the complement thereof, or a position corresponding to position 14,441 according to SEQ ID NO:3, or the complement thereof. When the sequenced portion of the HAL nucleic acid molecule in the biological sample comprises: an adenine at a position corresponding to position 11,352 according to SEQ ID NO:2, or the complement thereof, or a guanine at a position corresponding to position 14,441 according to SEQ ID NO:3, or the complement thereof; then the HAL nucleic acid molecule in the biological sample is a HAL variant nucleic acid molecule encoding a HAL predicted gain-of-function polypeptide.

In some embodiments, the assay comprises: a) contacting the biological sample with a primer hybridizing to a portion of the nucleotide sequence of the HAL genomic nucleic acid molecule, or the complement thereof, that is proximate to a position corresponding to position 11,352 according to SEQ ID NO:2, or the complement thereof, or proximate to a position corresponding to position 14,441 according to SEQ ID NO:3, or the complement thereof; b) extending the primer at least through the position of the nucleotide sequence of the HAL genomic nucleic acid molecule, or the complement thereof, corresponding to position 11,352 according to SEQ ID NO:2, or the complement thereof, or corresponding to position 14,441 according to SEQ ID NO:3, or the complement thereof; and c) determining whether the extension product of the primer comprises: an adenine at a position corresponding to position 11,352 according to SEQ ID NO:2, or the complement thereof, or a guanine at a position corresponding to position 14,441 according to SEQ ID NO:3, or the complement thereof.

In some embodiments, the assay comprises sequencing the entire nucleic acid molecule. In some embodiments, only a HAL genomic nucleic acid molecule is analyzed.

In some embodiments, the assay comprises: a) amplifying at least a portion of the HAL nucleic acid molecule, or the complement thereof, in the biological sample, wherein the amplified portion comprises: an adenine at a position corresponding to position 11,352 according to SEQ ID NO:2, or the complement thereof, or a guanine at a position corresponding to position 14,441 according to SEQ ID NO:3, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: an adenine at a position corresponding to position 11,352 according to SEQ ID NO:2, or the complement thereof, or a guanine at a position corresponding to position 14,441 according to SEQ ID NO:3, or the complement thereof; and d) detecting the detectable label.

In some embodiments, the assay comprises: a) amplifying at least a portion of the HAL genomic nucleic acid molecule, or the complement thereof, in the biological sample, wherein the portion comprises: an adenine at a position corresponding to position 11,352 according to SEQ ID NO:2, or the complement thereof, or a guanine at a position corresponding to position 14,441 according to SEQ ID NO:3, or the complement thereof; and b) detecting the detectable label.

In some embodiments, the assay comprises: contacting the HAL nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the HAL nucleic acid molecule, or the complement thereof, comprising: an adenine at a position corresponding to position 11,352 according to SEQ ID NO:2, or the complement thereof, or a guanine at a position corresponding to position 14,441 according to SEQ ID NO:3, or the complement thereof; and detecting the detectable label.

In some embodiments, the HAL nucleic acid molecule is present within a cell obtained from the subject.

Alteration-specific polymerase chain reaction techniques can be used to detect mutations such as SNPs in a nucleotide sequence. Alteration-specific primers can be used because the DNA polymerase will not extend when a mismatch with the template is present.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a HAL variant genomic nucleic acid molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether a HAL nucleic acid molecule (e.g., genomic nucleic acid molecule), or complement thereof, within a biological sample comprises a nucleotide sequence comprising an adenine at a position corresponding to position 11,352 according to SEQ ID NO:2, or the complement thereof, the biological sample can be subjected to an amplification method using a primer pair that includes a first primer derived from the 5′ flanking sequence adjacent to an adenine at a position corresponding to position 11,352 according to SEQ ID NO:2, and a second primer derived from the 3′ flanking sequence adjacent to an adenine at a position corresponding to position 11,352 according to SEQ ID NO:2 to produce an amplicon that is indicative of the presence of the SNP at a position encoding an adenine at a position corresponding to position 11,352 according to SEQ ID NO:2. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising an adenine at a position corresponding to position 11,352 according to SEQ ID NO:2, and at least 1, 2, 3, 4, 5, 6, 7, 8, 9, 10, or more nucleotides on each side of positions comprising an adenine at a position corresponding to position 11,352 according to SEQ ID NO:2.

In some embodiments, to determine whether a HAL nucleic acid molecule (e.g., genomic nucleic acid molecule), or complement thereof, within a biological sample comprises a nucleotide sequence comprising a guanine at a position corresponding to position 14,441 according to SEQ ID NO:3, or the complement thereof, the biological sample can be subjected to an amplification method using a primer pair that includes a first primer derived from the 5′ flanking sequence adjacent to a guanine at a position corresponding to position 14,441 according to SEQ ID NO:3, and a second primer derived from the 3′ flanking sequence adjacent to a guanine at a position corresponding to position 14,441 according to SEQ ID NO:3 to produce an amplicon that is indicative of the presence of the SNP at a position encoding a guanine at a position corresponding to position 14,441 according to SEQ ID NO:3. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a guanine at a position corresponding to position 14,431 according to SEQ ID NO:3, and at least 1, 2, 3, 4, 5, 6, 7, 8, 9, 10, or more nucleotides on each side of positions comprising a guanine at a position corresponding to position 14,441 according to SEQ ID NO:3.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of a HAL predicted gain-of-function polypeptide comprising performing an assay on a biological sample obtained from the subject to determine whether a HAL polypeptide in the biological sample contains one or more variations that causes the polypeptide to have a gain-of-function (partial or complete) or predicted gain-of-function (partial or complete).

In some embodiments, when the subject does not have a HAL predicted gain-of-function polypeptide, the subject has an increased risk of developing skin cancer, such as non-melanoma skin cancer, basal cell carcinoma, squamous cell carcinoma, melanoma, Merkel cell carcinoma, dermatofibrosarcoma protuberans, or sebaceous carcinoma. In some embodiments, when the subject has a HAL predicted gain-of-function polypeptide, the subject has a decreased risk of developing skin cancer, such as non-melanoma skin cancer, basal cell carcinoma, squamous cell carcinoma, melanoma, Merkel cell carcinoma, dermatofibrosarcoma protuberans, or sebaceous carcinoma.

The present disclosure also provides isolated nucleic acid molecules that hybridize to HAL variant genomic nucleic acid molecules. In some embodiments, such isolated nucleic acid molecules hybridize to HAL variant nucleic acid molecules under stringent conditions. Such nucleic acid molecules can be used, for example, as probes, primers, alteration-specific probes, or alteration-specific primers as described or exemplified herein.

In some embodiments, the isolated nucleic acid molecules hybridize to a portion of the HAL variant nucleic acid molecule that includes a position corresponding to position 11,352 according to SEQ ID NO:2, or position 14,441 according to SEQ ID NO:3.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the isolated alteration-specific probe or alteration-specific primer comprises at least about 15 nucleotides, wherein the alteration-specific probe or alteration-specific primer comprises a nucleotide sequence which is complementary to the nucleotide sequence of a portion of a HAL variant nucleic acid molecule encoding a HAL predicted gain-of-function polypeptide, or the complement thereof. In some embodiments, the portion comprises a position corresponding to position 11,352 according to SEQ ID NO:2, or the complement thereof, or position 14,441 according to SEQ ID NO:3, or the complement thereof.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to a HAL variant genomic nucleic acid molecule. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence of a HAL variant nucleic acid molecule encoding a HAL predicted gain-of-function polypeptide, wherein the portion comprises a position corresponding to position 11,352 according to SEQ ID NO:2, or the complement thereof, or position 14,441 according to SEQ ID NO:3, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the HAL variant genomic nucleic acid molecules disclosed herein. The primers described herein can be used to amplify the HAL variant genomic nucleic acid molecules, or a fragment thereof.

The present disclosure also provides pairs of primers comprising any of the primers described above. For example, if one of the primers' 3′-ends hybridizes to a guanine at a position corresponding to position 11,352 according to SEQ ID NO:1 (rather than an adenine) in a particular HAL nucleic acid molecule, then the presence of the amplified fragment would indicate the presence of a HAL reference genomic nucleic acid molecule. Conversely, if one of the primers' 3′-ends hybridizes to an adenine at a position corresponding to position 11,352 according to SEQ ID NO:2 (rather than a guanine) in a particular HAL nucleic acid molecule, then the presence of the amplified fragment would indicate the presence of the HAL variant genomic nucleic acid molecule. In some embodiments, the nucleotide of the primer complementary to the adenine at a position corresponding to position 11,352 according to SEQ ID NO:2 can be at the 3′ end of the primer.

The present disclosure also provides pairs of primers comprising any of the primers described above. For example, if one of the primers' 3′-ends hybridizes to an adenine at a position corresponding to position 14,441 according to SEQ ID NO:1 (rather than a guanine) in a particular HAL nucleic acid molecule, then the presence of the amplified fragment would indicate the presence of a HAL reference genomic nucleic acid molecule. Conversely, if one of the primers' 3′-ends hybridizes to a guanine at a position corresponding to position 14,441 according to SEQ ID NO:3 (rather than an adenine) in a particular HAL nucleic acid molecule, then the presence of the amplified fragment would indicate the presence of the HAL variant genomic nucleic acid molecule. In some embodiments, the nucleotide of the primer complementary to the guanine at a position corresponding to position 14,441 according to SEQ ID NO:3 can be at the 3′ end of the primer.

In the context of the present disclosure “specifically hybridizes” means that the probe or primer (such as, for example, the alteration-specific probe or alteration-specific primer) does not hybridize to a nucleotide sequence encoding a HAL reference genomic nucleic acid molecule.

In any of the embodiments described throughout the present disclosure, the probes (such as, for example, an alteration-specific probe) can comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well. In some embodiments, the support is a microarray.

The present disclosure also provides molecular complexes comprising or consisting of any of the HAL variant genomic nucleic acid molecules, or complement thereof, described herein and any of the alteration-specific primers or alteration-specific probes described herein. In some embodiments, the HAL variant genomic nucleic acid molecules, or complement thereof, in the molecular complexes are single-stranded. In some embodiments, the molecular complex comprises or consists of any of the HAL variant genomic nucleic acid molecules, or complement thereof, described herein and any of the alteration-specific primers described herein. In some embodiments, the molecular complex comprises or consists of any of the HAL variant genomic nucleic acid molecules, or complement thereof, described herein and any of the alteration-specific probes described herein.

In some embodiments, the molecular complex comprises an alteration-specific primer or an alteration-specific probe hybridized to a HAL genomic nucleic acid molecule encoding a HAL predicted gain-of-function polypeptide, wherein the alteration-specific primer or the alteration-specific probe is hybridized to the HAL genomic nucleic acid molecule at a position corresponding to position 11,352 according to SEQ ID NO:2, or the complement thereof. In some embodiments, the molecular complex comprises an alteration-specific primer or an alteration-specific probe hybridized to a HAL genomic nucleic acid molecule encoding a HAL predicted gain-of-function polypeptide, wherein the alteration-specific primer or the alteration-specific probe is hybridized to the HAL genomic nucleic acid molecule at a position corresponding to position 14,441 according to SEQ ID NO:3, or the complement thereof.

In some embodiments, the genomic nucleic acid molecule in the molecular complex comprises SEQ ID NO:2. In some embodiments, the genomic nucleic acid molecule in the molecular complex comprises SEQ ID NO:3.

In some embodiments, the molecular complex comprises an alteration-specific probe or an alteration-specific primer comprising a label. In some embodiments, the label is a fluorescent label, a radiolabel, or biotin. In some embodiments, the molecular complex further comprises a non-human polymerase.

The nucleotide sequence of a HAL reference genomic nucleic acid molecule is set forth in SEQ ID NO:1 (ENSG00000084110.11 encompassing chr12:95,972,662-95,996,344 in the GRCh38/hg38 human genome assembly). Referring to SEQ ID NO:1, position 11,352 is a guanine. Referring to SEQ ID NO:1, position 14,441 is an adenine.

A HAL variant genomic nucleic acid molecule exists, wherein the guanine at position 11,352 is replaced with an adenine. The nucleotide sequence of this HAL variant genomic nucleic acid molecule is set forth in SEQ ID NO:2 (rs3819817).

Another HAL variant genomic nucleic acid molecule exists, wherein the adenine at position 14,41 is replaced with a guanine. The nucleotide sequence of this HAL variant genomic nucleic acid molecule is set forth in SEQ ID NO:3 (rs10859995).

The nucleotide sequence of a HAL reference mRNA molecule is set forth in SEQ ID NO:4. The nucleotide sequence of another HAL reference mRNA molecule is set forth in SEQ ID NO:5. The nucleotide sequence of another HAL reference mRNA molecule is set forth in SEQ ID NO:6. The nucleotide sequence of another HAL reference mRNA molecule is set forth in SEQ ID NO:7. The nucleotide sequence of another HAL reference mRNA molecule is set forth in SEQ ID NO:8. The nucleotide sequence of another HAL reference mRNA molecule is set forth in SEQ ID NO:9. The nucleotide sequence of another HAL reference mRNA molecule is set forth in SEQ ID NO:10. The nucleotide sequence of another HAL reference mRNA molecule is set forth in SEQ ID NO:11. The nucleotide sequence of another HAL reference mRNA molecule is set forth in SEQ ID NO:12. The nucleotide sequence of another HAL reference mRNA molecule is set forth in SEQ ID NO:13. The nucleotide sequence of another HAL reference mRNA molecule is set forth in SEQ ID NO:14.

The nucleotide sequence of a HAL reference cDNA molecule is set forth in SEQ ID NO:15. The nucleotide sequence of another HAL reference cDNA molecule is set forth in SEQ ID NO:16. The nucleotide sequence of another HAL reference cDNA molecule is set forth in SEQ ID NO:17. The nucleotide sequence of another HAL reference cDNA molecule is set forth in SEQ ID NO:18. The nucleotide sequence of another HAL reference cDNA molecule is set forth in SEQ ID NO:19. The nucleotide sequence of another HAL reference cDNA molecule is set forth in SEQ ID NO:20. The nucleotide sequence of another HAL reference cDNA molecule is set forth in SEQ ID NO:21. The nucleotide sequence of another HAL reference cDNA molecule is set forth in SEQ ID NO:22. The nucleotide sequence of another HAL reference cDNA molecule is set forth in SEQ ID NO:23. The nucleotide sequence of another HAL reference cDNA molecule is set forth in SEQ ID NO:24. The nucleotide sequence of another HAL reference cDNA molecule is set forth in SEQ ID NO:25.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, agonists, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The isolated nucleic acid molecules, or the complement thereof, can also be present within a host cell. In some embodiments, the host cell can comprise the vector that comprises any of the nucleic acid molecules described herein, or the complement thereof. In some embodiments, the nucleic acid molecule is operably linked to a promoter active in the host cell. In some embodiments, the promoter is an exogenous promoter. In some embodiments, the promoter is an inducible promoter. In some embodiments, the host cell is a bacterial cell, a yeast cell, an insect cell, or a mammalian cell. In some embodiments, the host cell is a bacterial cell. In some embodiments, the host cell is a yeast cell. In some embodiments, the host cell is an insect cell. In some embodiments, the host cell is a mammalian cell.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀ alkyl or C₂₋₁₀ alkenyl, and C₂₋₁₀ alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀ alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein, or vectors comprising the same. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:2, or SEQ ID NO:3). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a HAL variant genomic nucleic acid molecule comprising a nucleotide sequence encoding a HAL predicted gain-of-function polypeptide, wherein the nucleotide sequence comprises an adenine at a position corresponding to position 11,352 according to SEQ ID NO:2 means that if the nucleotide sequence of the HAL genomic nucleic acid molecule is aligned to the sequence of SEQ ID NO:2, the HAL sequence has an adenine residue at the position that corresponds to position 11,352 of SEQ ID NO:2. These phrases refer to a HAL variant genomic nucleic acid molecule encoding a HAL predicted gain-of-function polypeptide, wherein the genomic nucleic acid molecule has a nucleotide sequence that comprises an adenine residue that is homologous to the adenine residue at position 11,352 of SEQ ID NO:2.

As described herein, a position within a HAL variant genomic nucleic acid molecule that corresponds to position 11,352 according to SEQ ID NO:2, for example, can be identified by performing a sequence alignment between the nucleotide sequence of a particular HAL genomic nucleic acid molecule and the nucleotide sequence of SEQ ID NO:2. A variety of computational algorithms exist that can be used for performing a sequence alignment to identify a nucleotide position that corresponds to, for example, position 11,352 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequence of a HAL reference polypeptide is set forth in SEQ ID NO:26. Referring to SEQ ID NO:26, the HAL reference polypeptide is 657 amino acids in length.

The amino acid sequence of another HAL reference polypeptide is set forth in SEQ ID NO:27. Referring to SEQ ID NO:27, the HAL reference polypeptide is 449 amino acids in length.

The amino acid sequence of another HAL reference polypeptide is set forth in SEQ ID NO:28. Referring to SEQ ID NO:28, the HAL reference polypeptide is 591 amino acids in length.

The amino acid sequence of another HAL reference polypeptide is set forth in SEQ ID NO:29. Referring to SEQ ID NO:29, the HAL reference polypeptide is 219 amino acids in length.

The amino acid sequence of another HAL reference polypeptide is set forth in SEQ ID NO:30. Referring to SEQ ID NO:30, the HAL reference polypeptide is 167 amino acids in length.

Because the HAL variant nucleic acid molecules described herein comprise variations within the non-coding region of HAL, the HAL predicted gain-of-function polypeptide can be any of the foregoing HAL polypeptides that are encoded by any of the HAL variant nucleic acid molecules described herein.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or prevent skin cancer for use in the treatment or prevention of skin cancer (or for use in the preparation of a medicament for treating or preventing skin cancer) in a subject, wherein the subject has any of the HAL variant genomic nucleic acid molecules encoding a HAL predicted gain-of-function polypeptide described herein. The therapeutic agents that treat or prevent skin cancer can be any of the therapeutic agents that treat or prevent skin cancer described herein. The skin cancer can be any of non-melanoma skin cancer, basal cell carcinoma, squamous cell carcinoma, melanoma, Merkel cell carcinoma, dermatofibrosarcoma protuberans, and sebaceous carcinoma.

In some embodiments, the subject is identified as having a genomic nucleic acid molecule encoding a HAL predicted gain-of-function polypeptide, or the complement thereof, wherein the genomic nucleic acid molecule has a nucleotide sequence comprising an adenine at a position corresponding to position 11,352 according to SEQ ID NO:2, or the complement thereof.

In some embodiments, the subject is identified as having a genomic nucleic acid molecule encoding a HAL predicted gain-of-function polypeptide, or the complement thereof, wherein the genomic nucleic acid molecule has a nucleotide sequence comprising a guanine at a position corresponding to position 14,441 according to SEQ ID NO:3, or the complement thereof.

The present disclosure also provides HAL agonists for use in the treatment or prevention of skin cancer (or for use in the preparation of a medicament for treating or preventing skin cancer) in a subject, wherein the subject is heterozygous for any of the HAL variant genomic nucleic acid molecules encoding a HAL predicted gain-of-function polypeptides described herein, or wherein the subject is reference for a HAL genomic nucleic acid molecule. The HAL agonists can be any of the HAL agonists described herein. The skin cancer can be any of non-melanoma skin cancer, basal cell carcinoma, squamous cell carcinoma, melanoma, Merkel cell carcinoma, dermatofibrosarcoma protuberans, and sebaceous carcinoma.

In some embodiments, the subject is reference for a HAL genomic nucleic acid molecule.

In some embodiments, the subject is heterozygous for a genomic nucleic acid molecule encoding a HAL predicted gain-of-function polypeptide, or the complement thereof, wherein the genomic nucleic acid molecule has a nucleotide sequence comprising an adenine at a position corresponding to position 11,352 according to SEQ ID NO:2, or the complement thereof.

In some embodiments, the subject is heterozygous for a genomic nucleic acid molecule encoding a HAL predicted gain-of-function polypeptide, or the complement thereof, wherein the genomic nucleic acid molecule has a nucleotide sequence comprising a guanine at a position corresponding to position 14,441 according to SEQ ID NO:3, or the complement thereof.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Association of Skin Cancer with HAL GOF Variants

The exomes of 454,787 UKB study participants were sequenced, with 95.8% of targeted bases covered at a depth of 20× or greater, as previously described (Szustakowski, Advancing Human Genetics Research and Drug Discovery through Exome Sequencing of the UK Biobank. bioRxiv, 2021; and Van Hout et al., Nature, 2020). Twelve million variants were identified in 39 million base pairs across the coding regions of 18,659 genes (data not shown). Among the variants identified were 3,375,252 (median of 10,260 per individual) synonymous, 7,689,495 (9,284 per individual) missense and 889,957 (212 per individual) putative gain-of-function (pLOF) variants (data not shown), of which about half were observed only once in this dataset (singleton variants; data not shown).

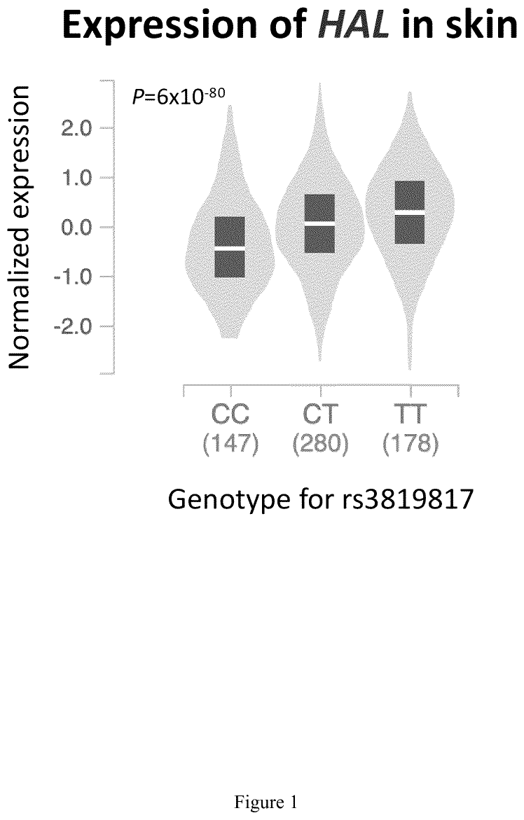

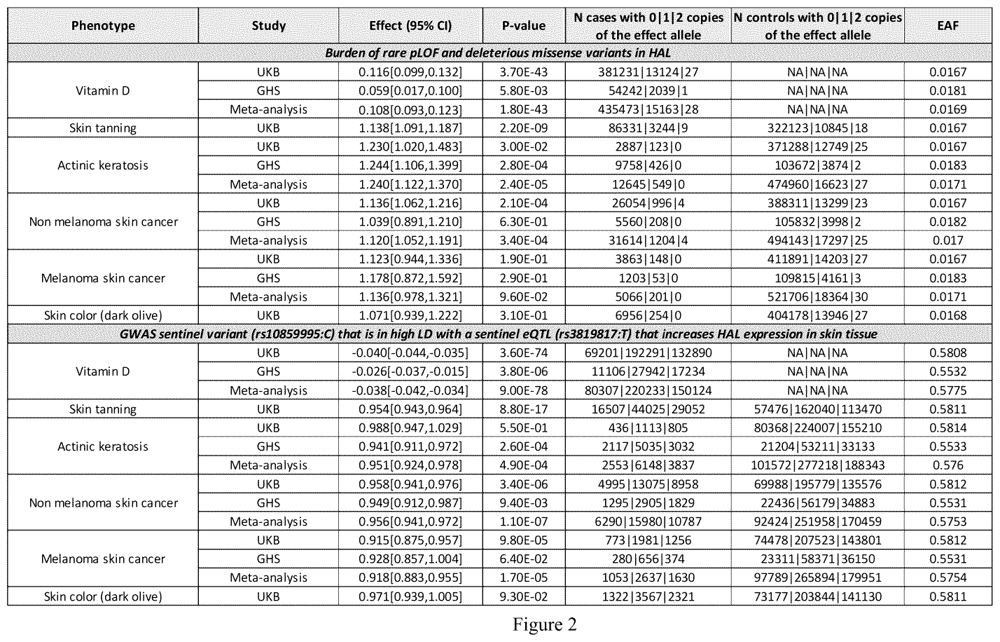

It was discovered that a burden of rare pLOF and deleterious missense variants in HAL was associated with higher vitamin D levels, as well as greater ease of skin tanning and higher risks of actinic keratosis and non-melanoma skin cancer (see, FIG. 2). These findings were independently supported by trait-lowering associations with a common variant (rs10859995:C, 58% frequency) that co-localizes (r²=0.97) with an expression quantitative trait locus (rs3819817:T) that increases HAL expression in skin tissue (G. T. Consortium, Science, 2020, 369, 1318-1330; see, FIG. 1). Altogether, these results implicate HAL in both vitamin D levels and skin cancer and highlight an allelic series that includes common gain-of-function non-coding variants (trait-lowering).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having skin cancer or at risk of developing skin cancer, the method comprising administering a Histidine Ammonia-Lyase (HAL) agonist to the subject.

2. The method according to claim 1, wherein the skin cancer comprises non-melanoma skin cancer, basal cell carcinoma, squamous cell carcinoma, melanoma, Merkel cell carcinoma, dermatofibrosarcoma protuberans, or sebaceous carcinoma.

3-8. (canceled)

9. The method according to claim 1, wherein the HAL agonist comprises HAL protein or thyroid hormone (T3).

10. The method according to claim 1, further comprising detecting the presence or absence of a HAL variant nucleic acid molecule encoding a HAL predicted gain-of-function polypeptide in a biological sample obtained from the subject.

11. The method according to claim 1, further comprising administering a therapeutic agent that treats or prevents skin cancer to the subject.

12. The method according to claim 10, further comprising administering a therapeutic agent that treats or prevents skin cancer in a dosage amount that is the same as or less than a standard dosage amount to a subject that is heterozygous for the HAL variant nucleic acid molecule.

13. The method according to claim 10, wherein the HAL variant nucleic acid molecule is a genomic nucleic acid molecule having a nucleotide sequence comprising: an adenine at a position corresponding to position 11,352 according to SEQ ID NO:2, or the complement thereof, or a guanine at a position corresponding to position 14,441 according to SEQ ID NO:3, or the complement thereof.

14. The method according to claim 10, wherein the detecting step is carried out in vitro.

15. The method according to claim 10, wherein the detecting step comprises sequencing at least a portion of the nucleotide sequence of the HAL genomic nucleic acid molecule, or the complement thereof, in the biological sample, wherein the sequenced portion comprises: a position corresponding to position 11,352 according to SEQ ID NO:2, or the complement thereof, or a position corresponding to position 14,441 according to SEQ ID NO:3, or the complement thereof,
wherein when the sequenced portion of the HAL genomic nucleic acid molecule in the biological sample comprises an adenine at a position corresponding to position 11,352 according to SEQ ID NO:2, or the complement thereof, or a guanine at a position corresponding to position 14,441 according to SEQ ID NO:3, or the complement thereof, then the HAL genomic nucleic acid molecule in the biological sample is a HAL variant genomic nucleic acid molecule encoding a HAL predicted gain-of-function polypeptide.

16. The method according to claim 10, wherein the detecting step comprises:
a) contacting the biological sample with a primer hybridizing to a portion of the nucleotide sequence of the HAL genomic nucleic acid molecule, or the complement thereof, that is proximate to a position corresponding to position 11,352 according to SEQ ID NO:2, or the complement thereof, or proximate to a position corresponding to position 14,441 according to SEQ ID NO:3, or the complement thereof,
b) extending the primer at least through the position of the nucleotide sequence of the HAL genomic nucleic acid molecule, or the complement thereof, corresponding to position 11,352 according to SEQ ID NO:2, or the complement thereof, or corresponding to position 14,441 according to SEQ ID NO:3, or the complement thereof, and
c) determining whether the extension product of the primer comprises an adenine at a position corresponding to position 11,352 according to SEQ ID NO:2, or the complement thereof, or comprises a guanine at a position corresponding to position 14,441 according to SEQ ID NO:3, or the complement thereof.

17. The method according to claim 15, wherein the detecting step comprises sequencing the entire nucleic acid molecule.

18. The method according to claim 10, wherein the detecting step comprises:
a) amplifying at least a portion of the HAL genomic nucleic acid molecule, or the complement thereof, in the biological sample, wherein the portion comprises: an adenine at a position corresponding to position 11,352 according to SEQ ID NO:2, or the complement thereof, or a guanine at a position corresponding to position 14,441 according to SEQ ID NO:3, or the complement thereof,
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleotide sequence of the amplified nucleic acid molecule comprising: an adenine at a position corresponding to position 11,352 according to SEQ ID NO:2, or the complement thereof, or a guanine at a position corresponding to position 14,441 according to SEQ ID NO:3, or the complement thereof, and
d) detecting the detectable label.

19. The method according to claim 10, wherein the detecting step comprises:
contacting the HAL genomic nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the HAL genomic nucleic acid molecule, or the complement thereof, comprising: an adenine at a position corresponding to position 11,352 according to SEQ ID NO:2, or the complement thereof, or a guanine at a position corresponding to position 14,441 according to SEQ ID NO:3, or the complement thereof, and
detecting the detectable label.

20-30. (canceled)

31. A method of identifying a subject having an increased risk of developing skin cancer, the method comprising:
determining or having determined the presence or absence of a Histidine Ammonia-Lyase (HAL) variant nucleic acid molecule encoding a HAL predicted gain-of-function polypeptide in a biological sample obtained from the subject;
wherein:
when the subject is HAL reference, then the subject has an increased risk of developing skin cancer; and
when the subject is heterozygous or homozygous for a HAL variant nucleic acid molecule encoding the HAL predicted gain-of-function polypeptide, then the subject has a decreased risk of developing skin cancer.

32-43. (canceled)

44. The method according to claim 1, wherein the subject is HAL reference.

45. The method according to claim 1, wherein the subject is heterozygous for a HAL variant nucleic acid molecule encoding a HAL predicted gain-of-function polypeptide.


SEQUENCE LISTING (30 sequences)

SEQ ID NO: 1 - DNA, 23683 bp, Homo sapiens
  source 1..23683: mol_type = genomic DNA
agataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcaggtacctcaataaatatttgtccaatgcatgaatgagtaaaggtgaaggagggggagtggtttgaaagtctattctcatccttccagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtgggtgagtggctgcaggagagggccgcgaacgggtgcggtttggcctggcttagtctttgcagcattgaacccccacctcagggaaaatgatgagcagaggtcaggcttgccgaggctggccctgtgcaggcacatgccacgcctctggggatcttaagtcctcacacaaccctgtgagatgggtaggattctcctccacctctgatggaggagaaatcagagaggaggtaacttacccagactccagcagctagtaagtaacaaagactagactggtactcaaggctgcaggtctgaatccctttgcagtgactgaatcattttgaggttattttgcctttcgaaagaatccacccccacccccagactggaggccggcagcatagcatcagttcagattttgaagaaaataaagctgggaatggggatggaagtggaattctgtcttgaactttcatctaaacccctggtctctgccctgacacctacctttctgctttttgctctcctcctacataaagatgctaagaccacatgaaaccatctgaagcagttttaaagcagccatttctatagaattcatgggccaatgatgggccgttccttggtttaacagggggaacatggctgtacaatgtgatctgtaacgagtctcccgtgcattgttttagttatagagggtgatgccatgtctcctgacttcattccatctcaaccagaaggagtttatctgtatcctttcccacaggctctttggtctgctctgggggtggctcctgcagatggtggggctgggggtttttccacaccaccctatatgttcctttacctggatacagatacagcaagtaccgggagcctgaaaaggtaagcttcaaaccactttctttttcttcagaaaattaaggccccttatttaagagggaggaaagccaggaaaacttctgagaaaacactgtcaattagaacagggctggccgggagcagtgactcatgtctgtagtcccagcactttgggtggccgaggtgggcagttcacttgaggtcatcagtttgaaaccagcctggccaacatggtgaaaccccatctctactaaaagtacaaaaattagccaggtatggtggcgtgcacctgtaataccagctactcaggagcctgaggcacgagaattgcttgaacccaggaggcagaggttgcagtgagccatgtttgtaccactgcactccagcctgggctggagactctgcctcaaaacaaacaaaccaaccaaccacagggctttatccagctctgtagtggggaaagggggcttggaaaggcattatgcttttgcaccaataagcctcctcttatgcaagatgcatttttatttagcatattcatgtttcacgcaatgatcagatctctgaaatgtcagtttggttctgttggaggtgtccctactctggttagaataatcaagctgttcagactcactgagatccccttcttttgtgtagtacatcgagttagatggagaccgtctgaccacggaggatctggtcaacttgggaaagggacgctacaaaataaaggtatggggagggaggggaaagatgtcctctggccatttttgttgcccgtggaagtctttaacgtgctcagttctgatcaacattttcctagctcaccccaacagctgagaagagggtgcagaaatccagggaggtcatagatagcatcataaaagagaaaacaggtatctttttatctttatgttataaatatttttataaacaaacaaaaatccctgcatttaattgcataatgtctctttttattctagttgtttacggtattactacaggttttgggaaatttgccagaactgtaattcctatcaataagctacagtaagtttaaaacacacatatgttcacgttccacctaccctcatcttttaaatatttttcaacaagggaaataattcacatttgccccaatttacagatcagaggatgaggcttgggttaccttgcatgtgagttggtattgaaaacccaggctggctggtttctcccattgccttccttctgtccctccaaggatttgaccatgattcccttcattttcccagctgagggaacattttctcactgcaatgtatctaagagggtttttacctttctgttgcagggagcttcaggtcaacttagtacgctcacattcttcaggtaagtcaaaacgttgggtgcctcctggatcttgtgtgattagatgaataaagcacagcacagtgtgagttatccagttatagtcacaaaaagctagtttctcaaaacagcatttcaagaactaggtgccgaggtgagaagggatgcagaaagaatcagctgagcctcagaaaaagaaatgggccaggtggagggatggggttggtatgagaacacatgtcccatgccatggagacactgttgcattcagggaaggaaataaagcctggaactcaaagaatgagttaggatgggcatagtgagatgggaaagactgtcaccaatcagcacccatcagatggtaaagaagttgtgctaaggactctggatgctattctaaaggtgagtgggaggtcaggggttcactgaagcttcccaaaaaggggaattgagaaggcaaaactggggtcttaacctttgtcaggaagatggggctcagacaagtcagacaggcaggagggagagtagtccaactacctgagccgagaggagaaggtaatagcaggctgggctacatatgggaaagattggagggagggagggcaattgtcaggagacaaaaaggagtttgcttttcttggagaaaacatcaattctgttttgcaggtgttgggaaaccactaagtcctgagaggtgtcggatgctcttggctttaaggatcaatgtcttagccaaaggatacagtggcatttccctggagaccctcaaacaagtcatagaaatgtttaatggtaatgcaatggctccctagacggaacctctgtggaggctggacatgtaccacagggtagaattgattggccagatgctgaggaatcacctgagaagacatgaaataattgaaatgagtgggaaatgccgttggcaaaaagtaggatagttagatgggggaagaggagaaagtcgatcagagatgcttactcctatgaactttaacttattctgcaagaccctttccctagcatttgtcatcttcaagactagaacaggactcttaagtctgcccttacattcaagcctgcctagaagaaacttggattaagtggtacatgaatatggttagaaaaattgcatgatatcaatgtccatcccaaaggccaattcagtagttctggttggtgcctgggaatctgaaatcgtaatgtcccccaggtgatggccagacttgcacactaaaatccaacttcttgcaagggtagaacagaaatgcaaagaggttaatgactcccttgtaatcaagaagccatgtagtggcaggatatgagggaactcaactctggagctttcttcttggataggaaaattctaatgtggaaaccagatttgcagtgattggaggtattgtcccttctcacaggtaaggacaagccttctaggtcagtggagactcagaactctttgaggggaaggagagggaggagaagcagtgctgaggcagtcacaaacagcaaaacacattcttgagtttccatgttatggacgcaagtggcacgtgaataggatctgccaatgagtggagagcaatttcaatgagcagtctattgtatatacagggctgtgctgaacagactagtagtgattccaacacctatttttactcctaccttggtctgccatactatatagccctaaaatcccagatagtagcactccaaccttaaacccacctgtactttatgggggagtctaaggcacctctgagaattccccagcgtgaactatcagccagaagttggtctgtggatggcactgccatacccgactgacattatttaacagttggaaaaaatgctgctgtcaggaatctgattttatttcagaaaaatgaagcctcttttttggtaactttttgggaatttcttagaaaatggcacgtgttcattgataaggggcagtggagtgtagactgggtgactggcaaaatgaaaagactcagttgcctatgacttctcttcccctgggaaaccttgttaatgctttggtgcaccacctccttctggaacattttatattacttttccaatgtttccaaacattttccaatagctgtgaatggctaaacttggtcaaatgaatgtcctttaatttcatatttagataatttctaactatttttcctataataaacaatgaactgtaaaacaaaaacaaaaacaaaaaaaaacagctagttcctgatcacttcttcaagatatattgctaaaatttctacaagtggaagtactaggttgaattagagcacacagattttgagggctttgcatccagaagtgtcctccttccttcttcttatttatttaggagacaggatcctgctttgtcacccaggctggagtgcagtggcacaatctcggctcactgcaacctccacctcacgggttcaagtgattctcccatctcagccttcccagtagttgggactacaggcatgcaccaccatacttggctaatttttgtatgttttgcagagacagggtttcaccgtgttgcccaggctggtcttgaactcctgggctcaagtgatatgcccgcctcggcctcccaaagtgctgggattacaggcttgagccactgcgcctggcccttctttccttcttgctgactttccctccagccctcctttcctccctgtctcctctttacctttctctcttcttcctctccacccccactcctctttttctttctataacaattcatccaggtcccttacatattagctgggtttgaagaattgtctttccccaggtatctatgccataaagtgatagatattggaggcagggtgtttgcgggggcagtgggggcactcactggcaaagcaaggagagtcagaatgcagtactcttgaactaattgctgcctacttctctcccagcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtaagactcgtagcagcttctatctgcagcaattgccccaggttggacaccaactgtgcttgcatgcttcgtatcacctagtccttacaacagcccagtgagggagctcctactatccttactttacagatgagacagcgaaagcttagaagtcagactaggcaagactgttgttagcttctatactcggctgtcagccttctgggtttggtcatcaccttggtgaaaaggcataactgctactgttaatcccaactatttatcctgcacctacaacatttccagtgcctagcagggaggtcacacactctcacaattttaattacgagctggtctcacaggtgcagtggtatggctcatagcacttactatatggcttctggaaagcaggtttcatttttccctaagctcttaccctagtggctaatcaaaaaagcctgtttttgataaagctttggtgttttcttcatctgtgtctgaatttcattacagcagcataaagcatcatttaatgtatcatccctgatgaactgctgggagcttccctgatagtaagtgaaaggggtagagggccccagaagaggaaacagctaaggaaatagataatccttttggggatgcagcttctagatcatctgtcttccgtgcaggcttgtctttgattatttcactatggacagcgttttcctccgaccaccctcatacagcaatcaccctgacctttctaaacagcaaattcaagcatgttctggtgaaaacttgtaatgcttcccattgtcctcaggataaaggctaaacttaaagtggctttcaagggtcttgtttgtcacattagcccctaatcccttctatgtgtggttctgcattgcagaaactggaggttgcttggagaaggaatgttctgggcagagggtctggtacaaatacgttcttctctctcctatttctgcttctgaaacatcttctagggatcttttgaagccagaagttcaagaccagcctgggcaagaaagcgaggctccgcctctacataaaataattaaaaaattatcctggcacagtagcatgtgcctgtagtcccagctactcaggaggctgaggatcacttgagcccaggagttcaaggctgcagtgagctaggattgcaccactgtgctcgctctagcctgggttacagagacaggtgtctaaaaaataaaaagaaaaatagaaaagccctctaagaagcttccgtctccgctgctccacttcctacctctcgagttccttgtgaccctcctgtatgctctcctagcaaatgattgttttccactgcaccccacccacttcccacatcctcaagcactgaatgtactattactcagattgccctgagcttgcctgtcttcattttgcctactcctagaccgaccccaacacccagaacagaatccagcctctagctgatacttgaatctgtgaaattgacgtagtaaatgggaccagctctgtccttctcttaccttaacttccccttccttctttcctagagagaccttaacttaatgactctctacttcttttctttcaagggaagattgttctgcccatcgccccctcgggattctgtctccatctagtagagggaattttataatcccctcttcattggtgctcacacatgtgccacaaaaaccctgctctgaggttttgccagtatttaaatgaagcactaatgaggttagaggactttttaggtcaagctctggaggcaagggagcgtgaactgggaggagtccagcctgagcacaacacagatgcatgtatgtgtgtcagatgcctgcatgtcaggaggcagtggtaaaaaaggtgaactgctaaggtagaaactgaacacgaaggacgtagaaaccaaaatttagggttgattctatagacaatggagcccctgatttcagtacaaaaattaattgaaagataagtctgctttcatgactaaagaacagaactagagaatagaccagagaacatgaatacagccagttcctgtaggtctcatttcttataagatggacttttgggcataaagagctattattggattccatatgatattggaaataggctagtatttcatttgatacccattttcatatttttaatcattctataggtgctagaagcccatggattgaaaccagttattttaaaaccaaaagaggtaaggaaactcaagaaaaatatgttcatagtgcatgagtttttatttatgcagtattttcagttttaatctcagcctcaatcattccttaaatgcaacttgcatgtttgcttaaagttcatccctgaagaaggggcaactccgtaagaatttaaaagattctttatgccaggcatggtggcggatgcctgtaatcctagcactttgggaagccgagacaggtggattgcctgagctcaggagtttgagaccagcctgggcaacatggtgaaacccatctctacccgccccccccgccccgccaaaaaagacaaaaattagccaggcatggtggcgcacacttgtaatcccagctaccctggaggctaaggtgggaggatcacttgagccctggaggtcaaggctgcagggagccatgatcgcaccactgcacaccagcctgagcaacacagtaagaccctgtctcaaaaaagaaaaaaagattcttcatgccacaatgcatagggggtctcttaggaacttaaaacttcaaatcgagttctgttaggatctctgagagatagatgttctcagcccaaattccttagaatgctctaagctaagagtcttggaccagaattgggtttttttggatgtctcatctataacagtaagaatttaaggaagaaatgcaacctaatgcactaaagattttatgtgcagtctacactccagagctgcattgctgctttgggaatgtggatacttaatgagtgtttcatttgccagatttgtgcttacgagttcacatatgatagcacttattcttcaagggcttaaataaacattactgtcccgcactttagaaaacataaacataattttttatgtttatgactggctaatttttctcttatgaaagcaaaagatacaaaatccacgggtatgtaggttcgttacaataatcacagtaggaaactttgctctctcccccgacagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactggtcagcagggttcttccttttggttgttattcattcaaccagaggtggtggggcggggtggctgagatctttagacacagaagtggtgggggcaggtagtagggaaggagaaaacccaagtgcctgtggcgtacccagtgtgctgcctcttcatctaggctatggcacaactgatctttgaaagaactgcaatttacagatacacacagcccggaagttgagtgacttgcctgagagcacccagggatctgggtgtgtcaagctctgtgctgtcttaggaatctttttacaggacttaggagtcctgtaaaaaggagtaggattagagttggtcccgactagaaaattggggaagaaaaactgtcgaattggtagcttatttagaaagtagtgaattttgacgacctcccaactataatatgaaaaagggcagaacattgtctaaaagtgaatgagttaaattaggccactgtagggctccctcctgttattcgccctgttcctttaaggtaggcccagtgtcttgagtggaattcccagtggtctcaacatatagtttgctctcagatatttaaagtagaaggagtcaagtcaataaggaaaatcgtgctttcggagttaagtttctaattctgagtggtggctaaggacccctgggacttttgagggctgtgtcagcaccgtagatggtcctgaaccattgatggtcatttgttctaatatcagaattactcgacctggcatggtggctcatgcctgtaatcccagcactttgggaggccaaggtaggaggatcgctagagcccaggagtttgaaaccagcctggggaacgtagtgagacccccatctttaaaaaaaataaataaatggaattattcagactttttctccttcatcaattctagacattcatgctcttcgacctcaccgtgggcaaattgaagttgcttttcggtttcggtcactcttggactcagatcaccacccatcagaaatagcaggtctgaccatgtttatgggagtgacctatttggttacgtgttttgtgaaaagaattggtaacatgatccctgtctcatcccctccacctcttccacagagagtcacaggttctgtgatcgcgtccaggatgcatacaccttgcgctgctgtccacaggtaaaataaagaaaaaaaaaaaggtcaataaaaaatgccaataaagtttttctttccccaggttcaggatgaaaaattttaaaataggaaagtgttagaaaaacatgtaatggtcaactatagtccaaatttcttactgtctctatgaaaagttatgtatattttacctttatgtgtgcaattgttgtaaagtttctattctatatggtgaactatttgttttcttttgattgttcaacctactttcagaggacaacctctttttttttttttttttttttttgagacagacaaccaacaaataacctctttgatttggagacagagtctcactctgtcaccctggctggagtacagtggcatgatctctgttcactacaatcttcacctcccaggttcaagtgattctcctgcctcaacctcccgagtagatgggattacaggcatgtgccaccatgcctaatttttgtatttttagtagagatggggtctcaccatgttggccaagctggtctcgaacccctgacctcaaatgatccacctgcctcagcctcccaaagtgctgggattacaggcgtgaggaccgtccccggcccagacaacctcttatgttgattcagtgccctaacttttaaaaattcttagtaataaaatcataaccagttagtagagttgtactgtttctccctgttaagatgaaactttaaccagatagtcagaggtgtttacaggaaagtcttaattatatcattttgaaactcaggtgcatagtaatggttataccttacttgcattttatgggcaatataaattattttaaatgaatttgtgatgcttaactttaaattaaaaatgcagatgacctgggttggcattaggcatctgtattacagatgtgtaatttacatatgacagttagagagccagtcatctcaataaaccatattttggtctgtgccaaatttcagtctatggtgtaatagagagtcctccctagaataaataccctcatttccctttagtcagcaagttatcttttctagctagcttattgagcaatagatcaaaacacctatgggtaagaattgccaaattagatctacaggaagattaagtcctgatgtaagacttccctgatattttaataacgatagcagattctatgtgacaggcaccattatcactgtctttcatttattaattcatttaatcttcccaactataagatactctttctcccattttgcaatggggaaactgaggcacagaattaagtatcttgcccacgatcacacagacagataattacttccttgacttctgggagaaaccagagccaggtgctgctttatagcattgtggctatttctggggtttgtacagcgtagctgccttatgtgtgagcatcttctaatgtgatgccttcgtgcttaatgaactttaccgccttggataatagctaccatttgagtaatctctgcgagtcacatctaagaatttgtttgcattattttatgtacttctcaaaagccataagtgctattgtctcttttctaagtcttcctatcttttaactataaacctaaggaacttgcccaagtcctaccactaacctgggagtgaatctgcctgtgtctagagttggcattcttcctcccacaccatgctgctttgtatagattgatctaatgtgaatatatatgataatgaatttattctaatttgtttcagtgaaaacccaaaagaattataacaaaattatcttcagttcaaataatttccatggttataactttaaaataagatcttctgtattctttataactttaagataagatcttctatattcttttaagaatattggtactcttcaggtaatcaaatgggtcatataccttattttgattgtattttaggtccatggtgtggtgaatgatacaatagcatttgtgaagaacatcattaccacagaactgaacagcgcaacagataatcctgtatcttttgattgtatactaacctgcaattatagaattgcagctggtaattttaggatctactgaaacaatcattttgtgtagcttctcagttccattgctccatttcacagagtaaatgaatcagtacctgaggctcagaggttaactgactttctccaggtcagagctggtaagtggcagaggtgacctgtgaacttactctcaggttccaagtctaacatgctgtactttcaaaatggacctattcaagatttaagaagatatttccagcattggctgacatgtttcctgaagacactggccctaaaacccactgccaaggttgttggtggaagggtgaagacgtgggtcacaaatgtgattgcagaaatgctgaacaatttatgcaggcctctctgctttgagtgagagcagtttgcattatgcagaggtcagccttttaacacttaccaaagtattgattaaagagttatggcttcctcatcctgaggtttttggcatttttttttttgtttatttttagacggagtctcactctgtctcctaggctggagtgcagtggtgccatctcggctctctgcaacctctgcctcacgggttcaagcgattctcctgcctcagcctcctgagtagctggaattacaggcatgtgccagcaggcctggctaatatttgtatttttagtagagacagggttttgctgtgttggccaggctggtctcaaactcctgacctcaagtgatccacctgcctcggcctcccaaagtgccgggattacaggtgtgagcgactacacccagcccccatcatgagtttttatttgtcatgcgcactttgctcaaattagtgcttttgcgtttgttcaaagaaatagtaaaactgattccatttctcaaggatggaggtcctctcttaatggtgtggcttatctcagagccatctggtttttctctggaatgttccatgatggctcagttccccagatgttgaggtgatcagtcaggagtcacactggcacagttttcatggagatagcactgcctctgaagataatcaaacgcctttggaatctatttttcctattccacatctgcctcagttctgaatcctagagaaagtgctgagtaagcagccccaaaagcaggtcacaggctgagttagaagatggttatgtgatagctccataaggtttcccatgagatggcttttgtatgtatgtgttttttgacaagcttacttcagaatgtgtgttttatgttctctcacattgttttacactgaagtcaaacccacactaaataagcctcagaaattgtgggtctctccattcacctcaataccaccccatcccccagatggaatagtagtttttatgaactctatgtagtcttatctcacgtaccagtcttttcactgtagggaagaggtatctattctacatttacatcattgcttttaaagggtatctcatagtttttagaaattaccattcaattactgaatatggggcaggggataattaattcctcactaaattcttctgctaccaattgcctcagcaagaatttcagccaaaagtcttttatttttttcaattaaagattgctaattattgataaatttccaataggctaatatttgggagttctcttgcaatactgctgttacctgaaagaatatgtgtctcaattttctgttgcagatacggagccccatcacacatgctggatattatgggagttaaattctaattgttttaatctaatagccaacaaacaaatttggaacaactagaggacaataaaaacatgtctaagttctaaattgatgcagaattccattgctatagggattcagagaatggagaagaggccaagaccaaggagctagacccaatgggctcacagttacacatgcagaacagagtaatccacacttgagtaggttaatgtaacacatacatctctaactctttcaaagttagaaactttcttgcacacagcaagttctcagttaataggtaatgaatattcttgagcccaagaggcaaagattagctctttagaaccaggaagaaggatctgttactgccttttcctgggtgcttgctggtttaagtaaagtgcatgatggaatagaattatctgaggctgggtttggtggctcacacctgtaatcccagcactttgggaggccaaggtgggctgatcacttgaggccaagagtccaagaccagcctggccaacatggcaaaaccctgtctctactaaaaatacaaaatttaactgggtgtgatggtgcatgcctgtaatcccagctactcaggaggctgaggcacaagaaatcacttgaacccgggaggcagaggtcgcagtgagccgagatggcgcccctgcactccagcctgggtgacagaatgatacagaaaataaaaaaagaattacctcagagtaataatattcagcaaatttaaacttgccacctacttaaaatggattcatgaggacatattggccaccatctgctgttgaacgctgacaccagattactgagtaaagaatttgaaaattttctcaaaattgattgttccagacattttggtagttgctcatcagcagagtttcccattcaacagattcttctatggaacacaaatttgcagtgcccattgaagaaacagaagttgctttcaaacagatgtgttggtctctgtttttagtttcaggctataaaccttttgagggcaggtactaaccaccaggttagtacagttatggtgctttagaatctaatctcaagagaaaacatcatttcaaggttcatgttttttcagcctccaaattgggtgtacatgatccacctttaaggctttttgttttttgcttttgtgcccctttatatctcttgcaggaagacttgtctctttccctccaccccacatttgtacacagactgccacctccacgttaaaaaagaaggcaggaaggggttgtacttgaagtgaccagcaaacattatcttcaagccttaacctcttttgaaagatggtctttgccaataggggagagacagtttctggaggaaacttccatggtgaatacccagccaaagtaagctttttaaaactgctcctgacccagaaggcacatttcaatataggctgactaaatggagaccctctttcaggccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttggtaaggacactgcccctttcccagaacacacgcccgtccatctaagggtctttccatttcacctagaagtatcaaatgtggtctgtgagtgagatctaacttttcaccacttttatctgcatcctagttctctcagtgcaaaggtaacaggttaactctaatatttttcagaaaaaaatagtttaaaaacttcgatactgttatgctgaaatataataatacaataataaggctgtaatggacaatttctctgtggaaagcaatttgacattgcatatgaacttcatctctatagcttttttccaaatctccatttgaagactattggaaaaagactaagatggttatagttaatggtgaaacttgaaccaacctataattaaactttgaacttttaaacctataattaaactttggattcatttcactagatataacaaccactaaaaacataaagatgttattgcaacattaaaaaaatgcttataagaatcaggataccctctgtgaagattgagcttcaatatagtttatttctgactcaagattgtaagagacagattaacacatcagaacagtgcctggcactaagtaagtactaagggcctcatttatgttcattcacttaattgcacaattgtcacctgaggtaagtgctattattgttatctatagatgaatatctgaagatctgagagttaagtgacttgcccaatattgcacagctagcaagatgtaaagcagaattcaaaccctgaattttggcatgcaaaaatgtgcttttacttcaattaatgaggatctcattttaatcaatacttaatcctaaaatagggttgtcaaatttaacaaaaacacaggatgactagttaaatttgaattatttgggacatacttgtactacaaaaattatttttttaatctaaaatcaaaatttaactgggcatcctttactttatctaacaaccctaccctaaaaggcaggatagcatgagttattacatgcgtagatgctagatcagaagcccagctctgccatttactagctctgtgagcctctttatcctgttgatcctcaatttacacctgcataaaatggagatcagtttacctaagggggttgtttggaggattaaatgaattgatgtatataaagcctggcacatcataagcactaaaaaaatactagttgctattaatattattcccaacgaacacaaataaccaactaagagccactgaattcacctcctctgaccggtatcttctatttgcttacttgcttattaataaaatacccctttaacaggtagaaatttctcacaataacgtagtagtgggggagtagtcattgatttctagagaacaggatggacattgggaagcctcatttgaatatgttttagtttcccaagctgcctattgaggttgtttggcttgctttcattgtagtaaaattagtgtttttctttttttggcccttgtaagttatctttccgaacaagccttaccacagtttacttatctgtatagtccacagtttagggtccaaggacttgggacttacagtgctgaaacctggaaagtccttggccaaccaacatgaccttgacaaagtagcatgaccctagtttaaagcgagatcatcttaggatgcgatcccatgcagcatagaaaacaactgaagcctcacccatgaccactcttaaagccagagaaagaggagaagagactttgagtctgacctttcctgacttctaagctagggtgagggaacttcataacctaagtttttgctacttatagtgtagtcctcagaccaacaatatggccatcacctgagagctcgttagaacagcagaatttcacgccccacccaaagctcagtatatttgttataacaaggtccctgagtgattcatgtgcactggaaagcttgagagtcccatgtcttggtcctccctagaattcccccacctcccagatatttctggcagtcatcgagcccctgtttacatccatttatgtcatccttgaacactctgaagtgttaaatcttcttgcccaagatttatgggtccagcctcagtccccaaatgccacatcggaattctggtgacacaaccaaattgacgtcaagtacctgaaaatggccatcatgtctctttctcccctagtctccctggcatgctatatgcttcccaggttgtcatcttggccttgttatattagaattcccagttctcccatctccgtcttacctcagaactcactattgctttagagtcctcacttggggaaaaaaaatgacctgcttccttttcccaaagcagctttctaccatcaggtgcttggataatgtccaagatcttctggagagtgtatcccatgctgtggagcactctgtggaagccacgggtcctttagacagctcatcctgtgaggagcacttcttaactggcactggtctcttgcagtttctgagaacaaggctctgtgccatccctcgtctgttgactccctctccaccagcgcagccacggaggaccacgtctccatgggaggatgggcagcaaggaaagccctcagggtcatcgagcatgtggagcaaggtaatgctgatgagttcggggtggcgggcctgcctgatagaccactgtgcccgtggttctcaagtgggatctcccaccagcaacatcagcatcacctggaaacttgttaggaaggcagactctcaggcctccttgcaggctggctaattcagacactccaggaggagagcggagcaatctgtgtttaacgaaccctccaggtgattctgatcttcactaaagattaagaaccaaggaaccatgcagaactaagctggtcctttggctggctttttttttctctttttttttttttttttttgaggcaggatctctatctgccacccaggctggagtgcagtgttgcaacaatagctcactgcagccttgaactcccaggctcaagtgatcctccttccttagtctcctaagtaactgacacctcaggcaagcaccaccacacctgggtaatttttaaattttttgtaaagatggggtcttgctgtgttgccttggctggtctcaaactcttggcctcaagcgatcctcccacctcacccccaaagtggttttttttttaatgagacttttaaaacatattcaaagtggagagaagagtaccatgaactctcacaccccaccttgattcaacagcacattcaatttatgaagtacactatgaactctagtgagtgcagacaggtcacagtgggagaggatgaagttgtggaattcacggtggacctggaagcaatttctcatcttctagtccagtatccccaccccgcaattcatatggaattggaaaccacgagatgtaaagcatcttgatgaaatgcatacaattatttaatagaactggggctcaagatgaggatcagctcttctttctgacttccttccctacccacatgacccaaacttggccaacaaacctttgttagaggatgtgaatgggggacatggcagattttgctagtgcagatgaatgggcagcttcctgggtaggtgtgcaggtctggtaattattcaggtaaatgtctcaattctccccttctcagcattaaagatatagtcaaggggttagaaaacaggttaaaatggcaactaaataccaaagcaacttcattctaccaatagacagagaacaaaccatctgttagttgggtggtgatgggtgtcaactttgggaaatccacattccttaagcatgaagtcagactttcataagctaccctcttggaatttcttgtttcagtgctggccatcgagctccttgcagcctgccagggcatagagtttctacgtcccctgaaaacaaccactccgctggagaaggtctatgacctggtgcgctctgttgtaaggtaagatcaaacaggtctctgaaaattacatcagttcagcactgtgaaccctggcagggtttcatgtttggctgatgggcggatgtgctctccctgtaggccctggataaaagatcgcttcatggccccggacatcgaggcagcccacaggctgctcctggagcagaaggcaagctggctccttggcttgtttcttaatttttaattgttacaggaaagcaaagtcggggatggagaaacattgccttattctattgttttctggccattccttgggaatgagcaaaaaggtatttgagcaacaacttgcagcaccactaagaagccatataataaacactaagtgcaccagggtgcagccaacggttgaatatctaaggattagaaacagaaatggattttaaatggagttttgggacaatattcatggggaaaaaaataagatgtcaggttcattgatgcctccccacccctgcaagatgtcctctttgattcaaaagtgcgttcccttctgggtgatgctatcacaactaccaccactgggtgcaatggctgcgtagacctggagagaaaaccaaggcccgggtgtttacttcagaagactttcggtctgtctagtgctctggtgagatgcgggtttttctttcttgcatatttccactggttacattgaccgtatactcagctgccaggtacagatgcccagcatgctggcttatcttttaaatatcgtttttgaagtgctttaaaccttttgaataacttgtgggtgttatgttccctagaatcgcttgatttccttaaaacagttgctctgctctggggcagtgatttttccctgtgattttcacacccattatttctaagcatgccctgcactctggatggggaagctgcttgcctgctgccaactttggtgctaggagggaggtatacgagaggttagcctttaagagtattcatttgctaaggtcataaaaaataataggaaaggctccttggctgtcagtggcaatcagttaaaaagcagatataaaccaaaaacagggccttaatttcattcaatttaaaaaaaaatttttttagagatgcgggtcttgctatgttgccaaggctggactcaaactcctgggctcaagtgatcctcccactttggcttcttgagtagctgggactacaggcacatgccaccatgcccgtccagggccttactttgagatgatgttatgcttaagaaaaaaaaaaaaaaagaaaaaagaggttggtgggtgggaggcaaaagctaaccaaggatctgaggcagtgcctctccaacattaaagtggctatgaatctgctgggcatcttgttaaaatgcaggtgtggattcagtaggtctgggtgaaggccaagattctgacttgtcccaggtgatgctgatactgcaggggcacaggccacgctttgagtagcagggtcaaaggaacagagcacctgtggcatggaaataaggcaatagggaaatacaattcactgccttttaccatttaaggcttgctttagaaaacacacaggtccaggccatgtgtggtggctcacgcctgtaatcccagcactttgggaagctgtggtgggtggatcacttgaggccagaaattcgagaccagcctggccaacatggtgaaaccccatctctgctaaaaatacaaaaattagctgggcctggtggcatgcacctgtaatcccagctactcaggaggctgaggcacaagaattgcttgaacctggaaggtggaggttgcagtgagctgagatcatgctactgcactccagcctgggcgacagagtgagactctgtctcacaaaaaaagaaaaaaaacaaaaaaacaagaaaaaaaccacataggtactgttaagaccctggagcaactatccttcactctatccacactgaattctctgggttttgttatttgcttgcttgttttgcttaggaaaagtatgcagcttaaaatctttatgatgactttagaaagttttggactttgtatatttacagtctttaatgcaaccaaagactaattgacacaggtcatgtgtcaaatggcttaaaataaaagcaagcaaacagcagctgtgtctggtcatgtatagagtatgttttgacttcggatgaagttgatgtttaatagcatgtggaaacaatgtcgtcaatatttcaactttgttttgtccttgcttttcaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataagtcctattgataaaggtcagatggagccctagagtgtattactgcatctatttttttccccgagaagataaaggaccttcagggatggcttaagtgtatctgtccagatgaaggatgggtcacatgacctcttggcttcccaagtctaagctctgtgactttgcaccagtgtgtgcatatatgtgcaaggcccttcaagtggtctgaaaccgtggctctaaaaaccacagctggtggagaggaggacagacacacttgccaccttgcctacctaattgccatctaaaatgggccgaacagtggatttcacaatagagttttcaccctttagatttacaacctgtcaggtggaaactgaagtgaaaactgctgcacacagcaattcagggagcaaaaaatgtgctgaggagactgtttacctaaaggttgttcttggtgctattccttgtcaaaatgtgaacacacacaaatgaggtttgtgcattgtcatccgtgggctgccattgagccagtaacccccagtggtctcatggtgctcttcgctccagtttggggaatgctggattctttcagcccctgcagccctccaggtcaaaatgacactttgtcactgagttttctacacagctctattagtaactgacagcacacgccttcaagggaacttcaagggaaacatggaataaactaagtctcaattgcc

SEQ ID NO: 2 - DNA, 23683 bp, Homo sapiens
  source 1..23683: mol_type = genomic DNA
agataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcaggtacctcaataaatatttgtccaatgcatgaatgagtaaaggtgaaggagggggagtggtttgaaagtctattctcatccttccagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtgggtgagtggctgcaggagagggccgcgaacgggtgcggtttggcctggcttagtctttgcagcattgaacccccacctcagggaaaatgatgagcagaggtcaggcttgccgaggctggccctgtgcaggcacatgccacgcctctggggatcttaagtcctcacacaaccctgtgagatgggtaggattctcctccacctctgatggaggagaaatcagagaggaggtaacttacccagactccagcagctagtaagtaacaaagactagactggtactcaaggctgcaggtctgaatccctttgcagtgactgaatcattttgaggttattttgcctttcgaaagaatccacccccacccccagactggaggccggcagcatagcatcagttcagattttgaagaaaataaagctgggaatggggatggaagtggaattctgtcttgaactttcatctaaacccctggtctctgccctgacacctacctttctgctttttgctctcctcctacataaagatgctaagaccacatgaaaccatctgaagcagttttaaagcagccatttctatagaattcatgggccaatgatgggccgttccttggtttaacagggggaacatggctgtacaatgtgatctgtaacgagtctcccgtgcattgttttagttatagagggtgatgccatgtctcctgacttcattccatctcaaccagaaggagtttatctgtatcctttcccacaggctctttggtctgctctgggggtggctcctgcagatggtggggctgggggtttttccacaccaccctatatgttcctttacctggatacagatacagcaagtaccgggagcctgaaaaggtaagcttcaaaccactttctttttcttcagaaaattaaggccccttatttaagagggaggaaagccaggaaaacttctgagaaaacactgtcaattagaacagggctggccgggagcagtgactcatgtctgtagtcccagcactttgggtggccgaggtgggcagttcacttgaggtcatcagtttgaaaccagcctggccaacatggtgaaaccccatctctactaaaagtacaaaaattagccaggtatggtggcgtgcacctgtaataccagctactcaggagcctgaggcacgagaattgcttgaacccaggaggcagaggttgcagtgagccatgtttgtaccactgcactccagcctgggctggagactctgcctcaaaacaaacaaaccaaccaaccacagggctttatccagctctgtagtggggaaagggggcttggaaaggcattatgcttttgcaccaataagcctcctcttatgcaagatgcatttttatttagcatattcatgtttcacgcaatgatcagatctctgaaatgtcagtttggttctgttggaggtgtccctactctggttagaataatcaagctgttcagactcactgagatccccttcttttgtgtagtacatcgagttagatggagaccgtctgaccacggaggatctggtcaacttgggaaagggacgctacaaaataaaggtatggggagggaggggaaagatgtcctctggccatttttgttgcccgtggaagtctttaacgtgctcagttctgatcaacattttcctagctcaccccaacagctgagaagagggtgcagaaatccagggaggtcatagatagcatcataaaagagaaaacaggtatctttttatctttatgttataaatatttttataaacaaacaaaaatccctgcatttaattgcataatgtctctttttattctagttgtttacggtattactacaggttttgggaaatttgccagaactgtaattcctatcaataagctacagtaagtttaaaacacacatatgttcacgttccacctaccctcatcttttaaatatttttcaacaagggaaataattcacatttgccccaatttacagatcagaggatgaggcttgggttaccttgcatgtgagttggtattgaaaacccaggctggctggtttctcccattgccttccttctgtccctccaaggatttgaccatgattcccttcattttcccagctgagggaacattttctcactgcaatgtatctaagagggtttttacctttctgttgcagggagcttcaggtcaacttagtacgctcacattcttcaggtaagtcaaaacgttgggtgcctcctggatcttgtgtgattagatgaataaagcacagcacagtgtgagttatccagttatagtcacaaaaagctagtttctcaaaacagcatttcaagaactaggtgccgaggtgagaagggatgcagaaagaatcagctgagcctcagaaaaagaaatgggccaggtggagggatggggttggtatgagaacacatgtcccatgccatggagacactgttgcattcagggaaggaaataaagcctggaactcaaagaatgagttaggatgggcatagtgagatgggaaagactgtcaccaatcagcacccatcagatggtaaagaagttgtgctaaggactctggatgctattctaaaggtgagtgggaggtcaggggttcactgaagcttcccaaaaaggggaattgagaaggcaaaactggggtcttaacctttgtcaggaagatggggctcagacaagtcagacaggcaggagggagagtagtccaactacctgagccgagaggagaaggtaatagcaggctgggctacatatgggaaagattggagggagggagggcaattgtcaggagacaaaaaggagtttgcttttcttggagaaaacatcaattctgttttgcaggtgttgggaaaccactaagtcctgagaggtgtcggatgctcttggctttaaggatcaatgtcttagccaaaggatacagtggcatttccctggagaccctcaaacaagtcatagaaatgtttaatggtaatgcaatggctccctagacggaacctctgtggaggctggacatgtaccacagggtagaattgattggccagatgctgaggaatcacctgagaagacatgaaataattgaaatgagtgggaaatgccgttggcaaaaagtaggatagttagatgggggaagaggagaaagtcgatcagagatgcttactcctatgaactttaacttattctgcaagaccctttccctagcatttgtcatcttcaagactagaacaggactcttaagtctgcccttacattcaagcctgcctagaagaaacttggattaagtggtacatgaatatggttagaaaaattgcatgatatcaatgtccatcccaaaggccaattcagtagttctggttggtgcctgggaatctgaaatcgtaatgtcccccaggtgatggccagacttgcacactaaaatccaacttcttgcaagggtagaacagaaatgcaaagaggttaatgactcccttgtaatcaagaagccatgtagtggcaggatatgagggaactcaactctggagctttcttcttggataggaaaattctaatgtggaaaccagatttgcagtgattggaggtattgtcccttctcacaggtaaggacaagccttctaggtcagtggagactcagaactctttgaggggaaggagagggaggagaagcagtgctgaggcagtcacaaacagcaaaacacattcttgagtttccatgttatggacgcaagtggcacgtgaataggatctgccaatgagtggagagcaatttcaatgagcagtctattgtatatacagggctgtgctgaacagactagtagtgattccaacacctatttttactcctaccttggtctgccatactatatagccctaaaatcccagatagtagcactccaaccttaaacccacctgtactttatgggggagtctaaggcacctctgagaattccccagcgtgaactatcagccagaagttggtctgtggatggcactgccatacccgactgacattatttaacagttggaaaaaatgctgctgtcaggaatctgattttatttcagaaaaatgaagcctcttttttggtaactttttgggaatttcttagaaaatggcacgtgttcattgataaggggcagtggagtgtagactgggtgactggcaaaatgaaaagactcagttgcctatgacttctcttcccctgggaaaccttgttaatgctttggtgcaccacctccttctggaacattttatattacttttccaatgtttccaaacattttccaatagctgtgaatggctaaacttggtcaaatgaatgtcctttaatttcatatttagataatttctaactatttttcctataataaacaatgaactgtaaaacaaaaacaaaaacaaaaaaaaacagctagttcctgatcacttcttcaagatatattgctaaaatttctacaagtggaagtactaggttgaattagagcacacagattttgagggctttgcatccagaagtgtcctccttccttcttcttatttatttaggagacaggatcctgctttgtcacccaggctggagtgcagtggcacaatctcggctcactgcaacctccacctcacgggttcaagtgattctcccatctcagccttcccagtagttgggactacaggcatgcaccaccatacttggctaatttttgtatgttttgcagagacagggtttcaccgtgttgcccaggctggtcttgaactcctgggctcaagtgatatgcccgcctcggcctcccaaagtgctgggattacaggcttgagccactgcgcctggcccttctttccttcttgctgactttccctccagccctcctttcctccctgtctcctctttacctttctctcttcttcctctccacccccactcctctttttctttctataacaattcatccaggtcccttacatattagctgggtttgaagaattgtctttccccaggtatctatgccataaagtgatagatattggaggcagggtgtttgcgggggcagtgggggcactcactggcaaagcaaggagagtcagaatgcagtactcttgaactaattgctgcctacttctctcccagcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtaagactcgtagcagcttctatctgcagcaattgccccaggttggacaccaactgtgcttgcatgcttcgtatcacctagtccttacaacagcccagtgagggagctcctactatccttactttacagatgagacagcgaaagcttagaagtcagactaggcaagactgttgttagcttctatactcggctgtcagccttctgggtttggtcatcaccttggtgaaaaggcataactgctactgttaatcccaactatttatcctgcacctacaacatttccagtgcctagcagggaggtcacacactctcacaattttaattacgagctggtctcacaggtgcagtggtatggctcatagcacttactatatggcttctggaaagcaggtttcatttttccctaagctcttaccctagtggctaatcaaaaaagcctgtttttgataaagctttggtgttttcttcatctgtgtctgaatttcattacagcagcataaagcatcatttaatgtatcatccctgatgaactgctgggagcttccctgatagtaagtgaaaggggtagagggccccagaagaggaaacagctaaggaaatagataatccttttggggatgcagcttctagatcatctgtcttccgtgcaggcttgtctttgattatttcactatggacagcgttttcctccgaccaccctcatacagcaatcaccctgacctttctaaacagcaaattcaagcatgttctggtgaaaacttgtaatgcttcccattgtcctcaggataaaggctaaacttaaagtggctttcaagggtcttgtttgtcacattagcccctaatcccttctatgtgtggttctgcattgcagaaactggaggttgcttggagaaggaatgttctgggcagagggtctggtacaaatacgttcttctctctcctatttctgcttctgaaacatcttctagggatcttttgaagccagaagttcaagaccagcctgggcaagaaagcgaggctccgcctctacataaaataattaaaaaattatcctggcacagtagcatgtgcctgtagtcccagctactcaggaggctgaggatcacttgagcccaggagttcaaggctgcagtgagctaggattgcaccactgtgctcgctctagcctgggttacagagacaggtgtctaaaaaataaaaagaaaaatagaaaagccctctaagaagcttccgtctccgctgctccacttcctacctctcgagttccttgtgaccctcctgtatgctctcctagcaaatgattgttttccactgcaccccacccacttcccacatcctcaagcactgaatgtactattactcagattgccctgagcttgcctgtcttcattttgcctactcctagaccgaccccaacacccagaacagaatccagcctctagctgatacttgaatctgtgaaattgacgtagtaaatgggaccagctctgtccttctcttaccttaacttccccttccttctttcctagagagaccttaacttaatgactctctacttcttttctttcaagggaagattgttctgcccatcgccccctcgggattctgtctccatctagtagagggaattttataatcccctcttcattggtgctcacacatgtgccacaaaaaccctgctctgaggttttgccagtatttaaatgaagcactaatgaggttagaggactttttaggtcaagctctggaggcaagggagcgtgaactgggaggagtccagcctgagcacaacacagatgcatgtatgtgtgtcagatgcctgcatgtcaggaggcagtggtaaaaaaggtgaactgctaaggtagaaactgaacacgaaggacgtagaaaccaaaatttagggttgattctatagacaatggagcccctgatttcagtacaaaaattaattgaaagataagtctgctttcatgactaaagaacagaactagagaatagaccagagaacatgaatacagccagttcctgtaggtctcatttcttataagatggacttttgggcataaagagctattattggattccatatgatattggaaataggctagtatttcatttgatacccattttcatatttttaatcattctataggtgctagaagcccatggattgaaaccagttattttaaaaccaaaagaggtaaggaaactcaagaaaaatatgttcatagtgcatgagtttttatttatgcagtattttcagttttaatctcagcctcaatcattccttaaatgcaacttgcatgtttgcttaaagttcatccctgaagaaggggcaactccgtaagaatttaaaagattctttatgccaggcatggtggcggatgcctgtaatcctagcactttgggaagccgagacaggtggattgcctgagctcaggagtttgagaccagcctgggcaacatggtgaaacccatctctacccgccccccccgccccgccaaaaaagacaaaaattagccaggcatggtggcgcacacttgtaatcccagctaccctggaggctaaggtgggaggatcacttgagccctggaggtcaaggctgcagggagccatgatcgcaccactgcacaccagcctgagcaacacagtaagaccctgtctcaaaaaagaaaaaaagattcttcatgccacaatgcatagggggtctcttaggaacttaaaacttcaaatcgagttctgttaggatctctgagagatagatgttctcagcccaaattccttagaatgctctaagctaagagtcttggaccagaattgggtttttttggatgtctcatctataacagtaagaatttaaggaagaaatgcaacctaatgcactaaagattttatgtgcagtctacactccagagctgcattgctgctttgggaatgtggatacttaatgagtgtttcatttgccagatttgtgcttacgagttcacatatgatagcacttattcttcaagggcttaaataaacattactgtcccgcactttagaaaacataaacataattttttatgtttatgactggctaatttttctcttatgaaagcaaaagatacaaaatccacgggtatgtaggttcgttacaataatcacagtaggaaactttgctctctcccccgacagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactggtcagcagggttcttccttttggttgttattcattcaaccagaggtggtggggcggggtggctgagatctttagacacagaagtggtgggggcaggtagtagggaaggagaaaacccaagtgcctgtggcgtacccagtgtgctgcctcttcatctaggctatggcacaactgatctttgaaagaactgcaatttacagatacacacagcccggaagttgagtgacttgcctgagagcacccagggatctgggtgtgtcaagctctgtgctgtcttaggaatctttttacaggacttaggagtcctgtaaaaaggagtaggattagagttggtcccgactagaaaattggggaagaaaaactgtcgaattggtagcttatttagaaagtagtgaattttgacgacctcccaactataatatgaaaaagggcagaacattgtctaaaagtgaatgagttaaattaggccactgtagggctccctcctgttattcgccctgttcctttaaggtaggcccagtgtcttgagtggaattcccagtggtctcaacatatagtttgctctcagatatttaaagtagaaggagtcaagtcaataaggaaaatcgtgctttcggagttaagtttctaattctgagtggtggctaaggacccctgggacttttgagggctgtgtcagcaccgtagatggtcctgaaccattgatggtcatttgttctaatatcagaattactcgacctggcatggtggctcatgcctgtaatcccagcactttgggaggccaaggtaggaggatcgctagagcccaggagtttgaaaccagcctggggaacgtagtgagacccccatctttaaaaaaaataaataaatggaattattcagactttttctccttcatcaattctagacattcatgctcttcgacctcaccgtgggcaaattgaagttgcttttcggtttcggtcactcttggactcagatcaccacccatcagaaatagcaggtctgaccatgtttatgggagtgacctatttggttacgtgttttgtgaaaagaattggtaacatgatccctgtctcatcccctccacctcttccacagagagtcacaggttctgtgatcgcgtccaggatgcatacaccttgcgctgctgtccacaggtaaaataaagaaaaaaaaaaaggtcaataaaaaatgccaataaagtttttctttccccaggttcaggatgaaaaattttaaaataggaaagtgttagaaaaacatgtaatggtcaactatagtccaaatttcttactgtctctatgaaaagttatgtatattttacctttatgtgtgcaattgttgtaaagtttctattctatatggtgaactatttgttttcttttgattgttcaacctactttcagaggacaacctctttttttttttttttttttttttgagacagacaaccaacaaataacctctttgatttggagacagagtctcactctgtcaccctggctggagtacagtggcatgatctctgttcactacaatcttcacctcccaggttcaagtgattctcctgcctcaacctcccgagtagatgggattacaggcatgtgccaccatgcctaatttttgtatttttagtagagatggggtctcaccatgttggccaagctggtctcgaacccctgacctcaaatgatccacctgcctcagcctcccaaagtgctgggattacaggcgtgaggaccgtccccggcccagacaacctcttatgttgattcagtgccctaacttttaaaaattcttagtaataaaatcataaccagttagtagagttgtactgtttctccctgttaagatgaaactttaaccagatagtcagaggtgtttacaggaaagtcttaattatatcattttgaaactcaggtgcatagtaatggttataccttacttgcattttatgggcaatataaattattttaaatgaatttgtgatgcttaactttaaattaaaaatgcagatgacctgggttggcattaggcatctgtattacagatgtgtaatttacatatgacaattagagagccagtcatctcaataaaccatattttggtctgtgccaaatttcagtctatggtgtaatagagagtcctccctagaataaataccctcatttccctttagtcagcaagttatcttttctagctagcttattgagcaatagatcaaaacacctatgggtaagaattgccaaattagatctacaggaagattaagtcctgatgtaagacttccctgatattttaataacgatagcagattctatgtgacaggcaccattatcactgtctttcatttattaattcatttaatcttcccaactataagatactctttctcccattttgcaatggggaaactgaggcacagaattaagtatcttgcccacgatcacacagacagataattacttccttgacttctgggagaaaccagagccaggtgctgctttatagcattgtggctatttctggggtttgtacagcgtagctgccttatgtgtgagcatcttctaatgtgatgccttcgtgcttaatgaactttaccgccttggataatagctaccatttgagtaatctctgcgagtcacatctaagaatttgtttgcattattttatgtacttctcaaaagccataagtgctattgtctcttttctaagtcttcctatcttttaactataaacctaaggaacttgcccaagtcctaccactaacctgggagtgaatctgcctgtgtctagagttggcattcttcctcccacaccatgctgctttgtatagattgatctaatgtgaatatatatgataatgaatttattctaatttgtttcagtgaaaacccaaaagaattataacaaaattatcttcagttcaaataatttccatggttataactttaaaataagatcttctgtattctttataactttaagataagatcttctatattcttttaagaatattggtactcttcaggtaatcaaatgggtcatataccttattttgattgtattttaggtccatggtgtggtgaatgatacaatagcatttgtgaagaacatcattaccacagaactgaacagcgcaacagataatcctgtatcttttgattgtatactaacctgcaattatagaattgcagctggtaattttaggatctactgaaacaatcattttgtgtagcttctcagttccattgctccatttcacagagtaaatgaatcagtacctgaggctcagaggttaactgactttctccaggtcagagctggtaagtggcagaggtgacctgtgaacttactctcaggttccaagtctaacatgctgtactttcaaaatggacctattcaagatttaagaagatatttccagcattggctgacatgtttcctgaagacactggccctaaaacccactgccaaggttgttggtggaagggtgaagacgtgggtcacaaatgtgattgcagaaatgctgaacaatttatgcaggcctctctgctttgagtgagagcagtttgcattatgcagaggtcagccttttaacacttaccaaagtattgattaaagagttatggcttcctcatcctgaggtttttggcatttttttttttgtttatttttagacggagtctcactctgtctcctaggctggagtgcagtggtgccatctcggctctctgcaacctctgcctcacgggttcaagcgattctcctgcctcagcctcctgagtagctggaattacaggcatgtgccagcaggcctggctaatatttgtatttttagtagagacagggttttgctgtgttggccaggctggtctcaaactcctgacctcaagtgatccacctgcctcggcctcccaaagtgccgggattacaggtgtgagcgactacacccagcccccatcatgagtttttatttgtcatgcgcactttgctcaaattagtgcttttgcgtttgttcaaagaaatagtaaaactgattccatttctcaaggatggaggtcctctcttaatggtgtggcttatctcagagccatctggtttttctctggaatgttccatgatggctcagttccccagatgttgaggtgatcagtcaggagtcacactggcacagttttcatggagatagcactgcctctgaagataatcaaacgcctttggaatctatttttcctattccacatctgcctcagttctgaatcctagagaaagtgctgagtaagcagccccaaaagcaggtcacaggctgagttagaagatggttatgtgatagctccataaggtttcccatgagatggcttttgtatgtatgtgttttttgacaagcttacttcagaatgtgtgttttatgttctctcacattgttttacactgaagtcaaacccacactaaataagcctcagaaattgtgggtctctccattcacctcaataccaccccatcccccagatggaatagtagtttttatgaactctatgtagtcttatctcacgtaccagtcttttcactgtagggaagaggtatctattctacatttacatcattgcttttaaagggtatctcatagtttttagaaattaccattcaattactgaatatggggcaggggataattaattcctcactaaattcttctgctaccaattgcctcagcaagaatttcagccaaaagtcttttatttttttcaattaaagattgctaattattgataaatttccaataggctaatatttgggagttctcttgcaatactgctgttacctgaaagaatatgtgtctcaattttctgttgcagatacggagccccatcacacatgctggatattatgggagttaaattctaattgttttaatctaatagccaacaaacaaatttggaacaactagaggacaataaaaacatgtctaagttctaaattgatgcagaattccattgctatagggattcagagaatggagaagaggccaagaccaaggagctagacccaatgggctcacagttacacatgcagaacagagtaatccacacttgagtaggttaatgtaacacatacatctctaactctttcaaagttagaaactttcttgcacacagcaagttctcagttaataggtaatgaatattcttgagcccaagaggcaaagattagctctttagaaccaggaagaaggatctgttactgccttttcctgggtgcttgctggtttaagtaaagtgcatgatggaatagaattatctgaggctgggtttggtggctcacacctgtaatcccagcactttgggaggccaaggtgggctgatcacttgaggccaagagtccaagaccagcctggccaacatggcaaaaccctgtctctactaaaaatacaaaatttaactgggtgtgatggtgcatgcctgtaatcccagctactcaggaggctgaggcacaagaaatcacttgaacccgggaggcagaggtcgcagtgagccgagatggcgcccctgcactccagcctgggtgacagaatgatacagaaaataaaaaaagaattacctcagagtaataatattcagcaaatttaaacttgccacctacttaaaatggattcatgaggacatattggccaccatctgctgttgaacgctgacaccagattactgagtaaagaatttgaaaattttctcaaaattgattgttccagacattttggtagttgctcatcagcagagtttcccattcaacagattcttctatggaacacaaatttgcagtgcccattgaagaaacagaagttgctttcaaacagatgtgttggtctctgtttttagtttcaggctataaaccttttgagggcaggtactaaccaccaggttagtacagttatggtgctttagaatctaatctcaagagaaaacatcatttcaaggttcatgttttttcagcctccaaattgggtgtacatgatccacctttaaggctttttgttttttgcttttgtgcccctttatatctcttgcaggaagacttgtctctttccctccaccccacatttgtacacagactgccacctccacgttaaaaaagaaggcaggaaggggttgtacttgaagtgaccagcaaacattatcttcaagccttaacctcttttgaaagatggtctttgccaataggggagagacagtttctggaggaaacttccatggtgaatacccagccaaagtaagctttttaaaactgctcctgacccagaaggcacatttcaatataggctgactaaatggagaccctctttcaggccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttggtaaggacactgcccctttcccagaacacacgcccgtccatctaagggtctttccatttcacctagaagtatcaaatgtggtctgtgagtgagatctaacttttcaccacttttatctgcatcctagttctctcagtgcaaaggtaacaggttaactctaatatttttcagaaaaaaatagtttaaaaacttcgatactgttatgctgaaatataataatacaataataaggctgtaatggacaatttctctgtggaaagcaatttgacattgcatatgaacttcatctctatagcttttttccaaatctccatttgaagactattggaaaaagactaagatggttatagttaatggtgaaacttgaaccaacctataattaaactttgaacttttaaacctataattaaactttggattcatttcactagatataacaaccactaaaaacataaagatgttattgcaacattaaaaaaatgcttataagaatcaggataccctctgtgaagattgagcttcaatatagtttatttctgactcaagattgtaagagacagattaacacatcagaacagtgcctggcactaagtaagtactaagggcctcatttatgttcattcacttaattgcacaattgtcacctgaggtaagtgctattattgttatctatagatgaatatctgaagatctgagagttaagtgacttgcccaatattgcacagctagcaagatgtaaagcagaattcaaaccctgaattttggcatgcaaaaatgtgcttttacttcaattaatgaggatctcattttaatcaatacttaatcctaaaatagggttgtcaaatttaacaaaaacacaggatgactagttaaatttgaattatttgggacatacttgtactacaaaaattatttttttaatctaaaatcaaaatttaactgggcatcctttactttatctaacaaccctaccctaaaaggcaggatagcatgagttattacatgcgtagatgctagatcagaagcccagctctgccatttactagctctgtgagcctctttatcctgttgatcctcaatttacacctgcataaaatggagatcagtttacctaagggggttgtttggaggattaaatgaattgatgtatataaagcctggcacatcataagcactaaaaaaatactagttgctattaatattattcccaacgaacacaaataaccaactaagagccactgaattcacctcctctgaccggtatcttctatttgcttacttgcttattaataaaatacccctttaacaggtagaaatttctcacaataacgtagtagtgggggagtagtcattgatttctagagaacaggatggacattgggaagcctcatttgaatatgttttagtttcccaagctgcctattgaggttgtttggcttgctttcattgtagtaaaattagtgtttttctttttttggcccttgtaagttatctttccgaacaagccttaccacagtttacttatctgtatagtccacagtttagggtccaaggacttgggacttacagtgctgaaacctggaaagtccttggccaaccaacatgaccttgacaaagtagcatgaccctagtttaaagcgagatcatcttaggatgcgatcccatgcagcatagaaaacaactgaagcctcacccatgaccactcttaaagccagagaaagaggagaagagactttgagtctgacctttcctgacttctaagctagggtgagggaacttcataacctaagtttttgctacttatagtgtagtcctcagaccaacaatatggccatcacctgagagctcgttagaacagcagaatttcacgccccacccaaagctcagtatatttgttataacaaggtccctgagtgattcatgtgcactggaaagcttgagagtcccatgtcttggtcctccctagaattcccccacctcccagatatttctggcagtcatcgagcccctgtttacatccatttatgtcatccttgaacactctgaagtgttaaatcttcttgcccaagatttatgggtccagcctcagtccccaaatgccacatcggaattctggtgacacaaccaaattgacgtcaagtacctgaaaatggccatcatgtctctttctcccctagtctccctggcatgctatatgcttcccaggttgtcatcttggccttgttatattagaattcccagttctcccatctccgtcttacctcagaactcactattgctttagagtcctcacttggggaaaaaaaatgacctgcttccttttcccaaagcagctttctaccatcaggtgcttggataatgtccaagatcttctggagagtgtatcccatgctgtggagcactctgtggaagccacgggtcctttagacagctcatcctgtgaggagcacttcttaactggcactggtctcttgcagtttctgagaacaaggctctgtgccatccctcgtctgttgactccctctccaccagcgcagccacggaggaccacgtctccatgggaggatgggcagcaaggaaagccctcagggtcatcgagcatgtggagcaaggtaatgctgatgagttcggggtggcgggcctgcctgatagaccactgtgcccgtggttctcaagtgggatctcccaccagcaacatcagcatcacctggaaacttgttaggaaggcagactctcaggcctccttgcaggctggctaattcagacactccaggaggagagcggagcaatctgtgtttaacgaaccctccaggtgattctgatcttcactaaagattaagaaccaaggaaccatgcagaactaagctggtcctttggctggctttttttttctctttttttttttttttttttgaggcaggatctctatctgccacccaggctggagtgcagtgttgcaacaatagctcactgcagccttgaactcccaggctcaagtgatcctccttccttagtctcctaagtaactgacacctcaggcaagcaccaccacacctgggtaatttttaaattttttgtaaagatggggtcttgctgtgttgccttggctggtctcaaactcttggcctcaagcgatcctcccacctcacccccaaagtggttttttttttaatgagacttttaaaacatattcaaagtggagagaagagtaccatgaactctcacaccccaccttgattcaacagcacattcaatttatgaagtacactatgaactctagtgagtgcagacaggtcacagtgggagaggatgaagttgtggaattcacggtggacctggaagcaatttctcatcttctagtccagtatccccaccccgcaattcatatggaattggaaaccacgagatgtaaagcatcttgatgaaatgcatacaattatttaatagaactggggctcaagatgaggatcagctcttctttctgacttccttccctacccacatgacccaaacttggccaacaaacctttgttagaggatgtgaatgggggacatggcagattttgctagtgcagatgaatgggcagcttcctgggtaggtgtgcaggtctggtaattattcaggtaaatgtctcaattctccccttctcagcattaaagatatagtcaaggggttagaaaacaggttaaaatggcaactaaataccaaagcaacttcattctaccaatagacagagaacaaaccatctgttagttgggtggtgatgggtgtcaactttgggaaatccacattccttaagcatgaagtcagactttcataagctaccctcttggaatttcttgtttcagtgctggccatcgagctccttgcagcctgccagggcatagagtttctacgtcccctgaaaacaaccactccgctggagaaggtctatgacctggtgcgctctgttgtaaggtaagatcaaacaggtctctgaaaattacatcagttcagcactgtgaaccctggcagggtttcatgtttggctgatgggcggatgtgctctccctgtaggccctggataaaagatcgcttcatggccccggacatcgaggcagcccacaggctgctcctggagcagaaggcaagctggctccttggcttgtttcttaatttttaattgttacaggaaagcaaagtcggggatggagaaacattgccttattctattgttttctggccattccttgggaatgagcaaaaaggtatttgagcaacaacttgcagcaccactaagaagccatataataaacactaagtgcaccagggtgcagccaacggttgaatatctaaggattagaaacagaaatggattttaaatggagttttgggacaatattcatggggaaaaaaataagatgtcaggttcattgatgcctccccacccctgcaagatgtcctctttgattcaaaagtgcgttcccttctgggtgatgctatcacaactaccaccactgggtgcaatggctgcgtagacctggagagaaaaccaaggcccgggtgtttacttcagaagactttcggtctgtctagtgctctggtgagatgcgggtttttctttcttgcatatttccactggttacattgaccgtatactcagctgccaggtacagatgcccagcatgctggcttatcttttaaatatcgtttttgaagtgctttaaaccttttgaataacttgtgggtgttatgttccctagaatcgcttgatttccttaaaacagttgctctgctctggggcagtgatttttccctgtgattttcacacccattatttctaagcatgccctgcactctggatggggaagctgcttgcctgctgccaactttggtgctaggagggaggtatacgagaggttagcctttaagagtattcatttgctaaggtcataaaaaataataggaaaggctccttggctgtcagtggcaatcagttaaaaagcagatataaaccaaaaacagggccttaatttcattcaatttaaaaaaaaatttttttagagatgcgggtcttgctatgttgccaaggctggactcaaactcctgggctcaagtgatcctcccactttggcttcttgagtagctgggactacaggcacatgccaccatgcccgtccagggccttactttgagatgatgttatgcttaagaaaaaaaaaaaaaaagaaaaaagaggttggtgggtgggaggcaaaagctaaccaaggatctgaggcagtgcctctccaacattaaagtggctatgaatctgctgggcatcttgttaaaatgcaggtgtggattcagtaggtctgggtgaaggccaagattctgacttgtcccaggtgatgctgatactgcaggggcacaggccacgctttgagtagcagggtcaaaggaacagagcacctgtggcatggaaataaggcaatagggaaatacaattcactgccttttaccatttaaggcttgctttagaaaacacacaggtccaggccatgtgtggtggctcacgcctgtaatcccagcactttgggaagctgtggtgggtggatcacttgaggccagaaattcgagaccagcctggccaacatggtgaaaccccatctctgctaaaaatacaaaaattagctgggcctggtggcatgcacctgtaatcccagctactcaggaggctgaggcacaagaattgcttgaacctggaaggtggaggttgcagtgagctgagatcatgctactgcactccagcctgggcgacagagtgagactctgtctcacaaaaaaagaaaaaaaacaaaaaaacaagaaaaaaaccacataggtactgttaagaccctggagcaactatccttcactctatccacactgaattctctgggttttgttatttgcttgcttgttttgcttaggaaaagtatgcagcttaaaatctttatgatgactttagaaagttttggactttgtatatttacagtctttaatgcaaccaaagactaattgacacaggtcatgtgtcaaatggcttaaaataaaagcaagcaaacagcagctgtgtctggtcatgtatagagtatgttttgacttcggatgaagttgatgtttaatagcatgtggaaacaatgtcgtcaatatttcaactttgttttgtccttgcttttcaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataagtcctattgataaaggtcagatggagccctagagtgtattactgcatctatttttttccccgagaagataaaggaccttcagggatggcttaagtgtatctgtccagatgaaggatgggtcacatgacctcttggcttcccaagtctaagctctgtgactttgcaccagtgtgtgcatatatgtgcaaggcccttcaagtggtctgaaaccgtggctctaaaaaccacagctggtggagaggaggacagacacacttgccaccttgcctacctaattgccatctaaaatgggccgaacagtggatttcacaatagagttttcaccctttagatttacaacctgtcaggtggaaactgaagtgaaaactgctgcacacagcaattcagggagcaaaaaatgtgctgaggagactgtttacctaaaggttgttcttggtgctattccttgtcaaaatgtgaacacacacaaatgaggtttgtgcattgtcatccgtgggctgccattgagccagtaacccccagtggtctcatggtgctcttcgctccagtttggggaatgctggattctttcagcccctgcagccctccaggtcaaaatgacactttgtcactgagttttctacacagctctattagtaactgacagcacacgccttcaagggaacttcaagggaaacatggaataaactaagtctcaattgcc

SEQ ID NO: 3 - DNA, 23683 bp, Homo sapiens
  source 1..23683: mol_type = genomic DNA
agataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcaggtacctcaataaatatttgtccaatgcatgaatgagtaaaggtgaaggagggggagtggtttgaaagtctattctcatccttccagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtgggtgagtggctgcaggagagggccgcgaacgggtgcggtttggcctggcttagtctttgcagcattgaacccccacctcagggaaaatgatgagcagaggtcaggcttgccgaggctggccctgtgcaggcacatgccacgcctctggggatcttaagtcctcacacaaccctgtgagatgggtaggattctcctccacctctgatggaggagaaatcagagaggaggtaacttacccagactccagcagctagtaagtaacaaagactagactggtactcaaggctgcaggtctgaatccctttgcagtgactgaatcattttgaggttattttgcctttcgaaagaatccacccccacccccagactggaggccggcagcatagcatcagttcagattttgaagaaaataaagctgggaatggggatggaagtggaattctgtcttgaactttcatctaaacccctggtctctgccctgacacctacctttctgctttttgctctcctcctacataaagatgctaagaccacatgaaaccatctgaagcagttttaaagcagccatttctatagaattcatgggccaatgatgggccgttccttggtttaacagggggaacatggctgtacaatgtgatctgtaacgagtctcccgtgcattgttttagttatagagggtgatgccatgtctcctgacttcattccatctcaaccagaaggagtttatctgtatcctttcccacaggctctttggtctgctctgggggtggctcctgcagatggtggggctgggggtttttccacaccaccctatatgttcctttacctggatacagatacagcaagtaccgggagcctgaaaaggtaagcttcaaaccactttctttttcttcagaaaattaaggccccttatttaagagggaggaaagccaggaaaacttctgagaaaacactgtcaattagaacagggctggccgggagcagtgactcatgtctgtagtcccagcactttgggtggccgaggtgggcagttcacttgaggtcatcagtttgaaaccagcctggccaacatggtgaaaccccatctctactaaaagtacaaaaattagccaggtatggtggcgtgcacctgtaataccagctactcaggagcctgaggcacgagaattgcttgaacccaggaggcagaggttgcagtgagccatgtttgtaccactgcactccagcctgggctggagactctgcctcaaaacaaacaaaccaaccaaccacagggctttatccagctctgtagtggggaaagggggcttggaaaggcattatgcttttgcaccaataagcctcctcttatgcaagatgcatttttatttagcatattcatgtttcacgcaatgatcagatctctgaaatgtcagtttggttctgttggaggtgtccctactctggttagaataatcaagctgttcagactcactgagatccccttcttttgtgtagtacatcgagttagatggagaccgtctgaccacggaggatctggtcaacttgggaaagggacgctacaaaataaaggtatggggagggaggggaaagatgtcctctggccatttttgttgcccgtggaagtctttaacgtgctcagttctgatcaacattttcctagctcaccccaacagctgagaagagggtgcagaaatccagggaggtcatagatagcatcataaaagagaaaacaggtatctttttatctttatgttataaatatttttataaacaaacaaaaatccctgcatttaattgcataatgtctctttttattctagttgtttacggtattactacaggttttgggaaatttgccagaactgtaattcctatcaataagctacagtaagtttaaaacacacatatgttcacgttccacctaccctcatcttttaaatatttttcaacaagggaaataattcacatttgccccaatttacagatcagaggatgaggcttgggttaccttgcatgtgagttggtattgaaaacccaggctggctggtttctcccattgccttccttctgtccctccaaggatttgaccatgattcccttcattttcccagctgagggaacattttctcactgcaatgtatctaagagggtttttacctttctgttgcagggagcttcaggtcaacttagtacgctcacattcttcaggtaagtcaaaacgttgggtgcctcctggatcttgtgtgattagatgaataaagcacagcacagtgtgagttatccagttatagtcacaaaaagctagtttctcaaaacagcatttcaagaactaggtgccgaggtgagaagggatgcagaaagaatcagctgagcctcagaaaaagaaatgggccaggtggagggatggggttggtatgagaacacatgtcccatgccatggagacactgttgcattcagggaaggaaataaagcctggaactcaaagaatgagttaggatgggcatagtgagatgggaaagactgtcaccaatcagcacccatcagatggtaaagaagttgtgctaaggactctggatgctattctaaaggtgagtgggaggtcaggggttcactgaagcttcccaaaaaggggaattgagaaggcaaaactggggtcttaacctttgtcaggaagatggggctcagacaagtcagacaggcaggagggagagtagtccaactacctgagccgagaggagaaggtaatagcaggctgggctacatatgggaaagattggagggagggagggcaattgtcaggagacaaaaaggagtttgcttttcttggagaaaacatcaattctgttttgcaggtgttgggaaaccactaagtcctgagaggtgtcggatgctcttggctttaaggatcaatgtcttagccaaaggatacagtggcatttccctggagaccctcaaacaagtcatagaaatgtttaatggtaatgcaatggctccctagacggaacctctgtggaggctggacatgtaccacagggtagaattgattggccagatgctgaggaatcacctgagaagacatgaaataattgaaatgagtgggaaatgccgttggcaaaaagtaggatagttagatgggggaagaggagaaagtcgatcagagatgcttactcctatgaactttaacttattctgcaagaccctttccctagcatttgtcatcttcaagactagaacaggactcttaagtctgcccttacattcaagcctgcctagaagaaacttggattaagtggtacatgaatatggttagaaaaattgcatgatatcaatgtccatcccaaaggccaattcagtagttctggttggtgcctgggaatctgaaatcgtaatgtcccccaggtgatggccagacttgcacactaaaatccaacttcttgcaagggtagaacagaaatgcaaagaggttaatgactcccttgtaatcaagaagccatgtagtggcaggatatgagggaactcaactctggagctttcttcttggataggaaaattctaatgtggaaaccagatttgcagtgattggaggtattgtcccttctcacaggtaaggacaagccttctaggtcagtggagactcagaactctttgaggggaaggagagggaggagaagcagtgctgaggcagtcacaaacagcaaaacacattcttgagtttccatgttatggacgcaagtggcacgtgaataggatctgccaatgagtggagagcaatttcaatgagcagtctattgtatatacagggctgtgctgaacagactagtagtgattccaacacctatttttactcctaccttggtctgccatactatatagccctaaaatcccagatagtagcactccaaccttaaacccacctgtactttatgggggagtctaaggcacctctgagaattccccagcgtgaactatcagccagaagttggtctgtggatggcactgccatacccgactgacattatttaacagttggaaaaaatgctgctgtcaggaatctgattttatttcagaaaaatgaagcctcttttttggtaactttttgggaatttcttagaaaatggcacgtgttcattgataaggggcagtggagtgtagactgggtgactggcaaaatgaaaagactcagttgcctatgacttctcttcccctgggaaaccttgttaatgctttggtgcaccacctccttctggaacattttatattacttttccaatgtttccaaacattttccaatagctgtgaatggctaaacttggtcaaatgaatgtcctttaatttcatatttagataatttctaactatttttcctataataaacaatgaactgtaaaacaaaaacaaaaacaaaaaaaaacagctagttcctgatcacttcttcaagatatattgctaaaatttctacaagtggaagtactaggttgaattagagcacacagattttgagggctttgcatccagaagtgtcctccttccttcttcttatttatttaggagacaggatcctgctttgtcacccaggctggagtgcagtggcacaatctcggctcactgcaacctccacctcacgggttcaagtgattctcccatctcagccttcccagtagttgggactacaggcatgcaccaccatacttggctaatttttgtatgttttgcagagacagggtttcaccgtgttgcccaggctggtcttgaactcctgggctcaagtgatatgcccgcctcggcctcccaaagtgctgggattacaggcttgagccactgcgcctggcccttctttccttcttgctgactttccctccagccctcctttcctccctgtctcctctttacctttctctcttcttcctctccacccccactcctctttttctttctataacaattcatccaggtcccttacatattagctgggtttgaagaattgtctttccccaggtatctatgccataaagtgatagatattggaggcagggtgtttgcgggggcagtgggggcactcactggcaaagcaaggagagtcagaatgcagtactcttgaactaattgctgcctacttctctcccagcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtaagactcgtagcagcttctatctgcagcaattgccccaggttggacaccaactgtgcttgcatgcttcgtatcacctagtccttacaacagcccagtgagggagctcctactatccttactttacagatgagacagcgaaagcttagaagtcagactaggcaagactgttgttagcttctatactcggctgtcagccttctgggtttggtcatcaccttggtgaaaaggcataactgctactgttaatcccaactatttatcctgcacctacaacatttccagtgcctagcagggaggtcacacactctcacaattttaattacgagctggtctcacaggtgcagtggtatggctcatagcacttactatatggcttctggaaagcaggtttcatttttccctaagctcttaccctagtggctaatcaaaaaagcctgtttttgataaagctttggtgttttcttcatctgtgtctgaatttcattacagcagcataaagcatcatttaatgtatcatccctgatgaactgctgggagcttccctgatagtaagtgaaaggggtagagggccccagaagaggaaacagctaaggaaatagataatccttttggggatgcagcttctagatcatctgtcttccgtgcaggcttgtctttgattatttcactatggacagcgttttcctccgaccaccctcatacagcaatcaccctgacctttctaaacagcaaattcaagcatgttctggtgaaaacttgtaatgcttcccattgtcctcaggataaaggctaaacttaaagtggctttcaagggtcttgtttgtcacattagcccctaatcccttctatgtgtggttctgcattgcagaaactggaggttgcttggagaaggaatgttctgggcagagggtctggtacaaatacgttcttctctctcctatttctgcttctgaaacatcttctagggatcttttgaagccagaagttcaagaccagcctgggcaagaaagcgaggctccgcctctacataaaataattaaaaaattatcctggcacagtagcatgtgcctgtagtcccagctactcaggaggctgaggatcacttgagcccaggagttcaaggctgcagtgagctaggattgcaccactgtgctcgctctagcctgggttacagagacaggtgtctaaaaaataaaaagaaaaatagaaaagccctctaagaagcttccgtctccgctgctccacttcctacctctcgagttccttgtgaccctcctgtatgctctcctagcaaatgattgttttccactgcaccccacccacttcccacatcctcaagcactgaatgtactattactcagattgccctgagcttgcctgtcttcattttgcctactcctagaccgaccccaacacccagaacagaatccagcctctagctgatacttgaatctgtgaaattgacgtagtaaatgggaccagctctgtccttctcttaccttaacttccccttccttctttcctagagagaccttaacttaatgactctctacttcttttctttcaagggaagattgttctgcccatcgccccctcgggattctgtctccatctagtagagggaattttataatcccctcttcattggtgctcacacatgtgccacaaaaaccctgctctgaggttttgccagtatttaaatgaagcactaatgaggttagaggactttttaggtcaagctctggaggcaagggagcgtgaactgggaggagtccagcctgagcacaacacagatgcatgtatgtgtgtcagatgcctgcatgtcaggaggcagtggtaaaaaaggtgaactgctaaggtagaaactgaacacgaaggacgtagaaaccaaaatttagggttgattctatagacaatggagcccctgatttcagtacaaaaattaattgaaagataagtctgctttcatgactaaagaacagaactagagaatagaccagagaacatgaatacagccagttcctgtaggtctcatttcttataagatggacttttgggcataaagagctattattggattccatatgatattggaaataggctagtatttcatttgatacccattttcatatttttaatcattctataggtgctagaagcccatggattgaaaccagttattttaaaaccaaaagaggtaaggaaactcaagaaaaatatgttcatagtgcatgagtttttatttatgcagtattttcagttttaatctcagcctcaatcattccttaaatgcaacttgcatgtttgcttaaagttcatccctgaagaaggggcaactccgtaagaatttaaaagattctttatgccaggcatggtggcggatgcctgtaatcctagcactttgggaagccgagacaggtggattgcctgagctcaggagtttgagaccagcctgggcaacatggtgaaacccatctctacccgccccccccgccccgccaaaaaagacaaaaattagccaggcatggtggcgcacacttgtaatcccagctaccctggaggctaaggtgggaggatcacttgagccctggaggtcaaggctgcagggagccatgatcgcaccactgcacaccagcctgagcaacacagtaagaccctgtctcaaaaaagaaaaaaagattcttcatgccacaatgcatagggggtctcttaggaacttaaaacttcaaatcgagttctgttaggatctctgagagatagatgttctcagcccaaattccttagaatgctctaagctaagagtcttggaccagaattgggtttttttggatgtctcatctataacagtaagaatttaaggaagaaatgcaacctaatgcactaaagattttatgtgcagtctacactccagagctgcattgctgctttgggaatgtggatacttaatgagtgtttcatttgccagatttgtgcttacgagttcacatatgatagcacttattcttcaagggcttaaataaacattactgtcccgcactttagaaaacataaacataattttttatgtttatgactggctaatttttctcttatgaaagcaaaagatacaaaatccacgggtatgtaggttcgttacaataatcacagtaggaaactttgctctctcccccgacagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactggtcagcagggttcttccttttggttgttattcattcaaccagaggtggtggggcggggtggctgagatctttagacacagaagtggtgggggcaggtagtagggaaggagaaaacccaagtgcctgtggcgtacccagtgtgctgcctcttcatctaggctatggcacaactgatctttgaaagaactgcaatttacagatacacacagcccggaagttgagtgacttgcctgagagcacccagggatctgggtgtgtcaagctctgtgctgtcttaggaatctttttacaggacttaggagtcctgtaaaaaggagtaggattagagttggtcccgactagaaaattggggaagaaaaactgtcgaattggtagcttatttagaaagtagtgaattttgacgacctcccaactataatatgaaaaagggcagaacattgtctaaaagtgaatgagttaaattaggccactgtagggctccctcctgttattcgccctgttcctttaaggtaggcccagtgtcttgagtggaattcccagtggtctcaacatatagtttgctctcagatatttaaagtagaaggagtcaagtcaataaggaaaatcgtgctttcggagttaagtttctaattctgagtggtggctaaggacccctgggacttttgagggctgtgtcagcaccgtagatggtcctgaaccattgatggtcatttgttctaatatcagaattactcgacctggcatggtggctcatgcctgtaatcccagcactttgggaggccaaggtaggaggatcgctagagcccaggagtttgaaaccagcctggggaacgtagtgagacccccatctttaaaaaaaataaataaatggaattattcagactttttctccttcatcaattctagacattcatgctcttcgacctcaccgtgggcaaattgaagttgcttttcggtttcggtcactcttggactcagatcaccacccatcagaaatagcaggtctgaccatgtttatgggagtgacctatttggttacgtgttttgtgaaaagaattggtaacatgatccctgtctcatcccctccacctcttccacagagagtcacaggttctgtgatcgcgtccaggatgcatacaccttgcgctgctgtccacaggtaaaataaagaaaaaaaaaaaggtcaataaaaaatgccaataaagtttttctttccccaggttcaggatgaaaaattttaaaataggaaagtgttagaaaaacatgtaatggtcaactatagtccaaatttcttactgtctctatgaaaagttatgtatattttacctttatgtgtgcaattgttgtaaagtttctattctatatggtgaactatttgttttcttttgattgttcaacctactttcagaggacaacctctttttttttttttttttttttttgagacagacaaccaacaaataacctctttgatttggagacagagtctcactctgtcaccctggctggagtacagtggcatgatctctgttcactacaatcttcacctcccaggttcaagtgattctcctgcctcaacctcccgagtagatgggattacaggcatgtgccaccatgcctaatttttgtatttttagtagagatggggtctcaccatgttggccaagctggtctcgaacccctgacctcaaatgatccacctgcctcagcctcccaaagtgctgggattacaggcgtgaggaccgtccccggcccagacaacctcttatgttgattcagtgccctaacttttaaaaattcttagtaataaaatcataaccagttagtagagttgtactgtttctccctgttaagatgaaactttaaccagatagtcagaggtgtttacaggaaagtcttaattatatcattttgaaactcaggtgcatagtaatggttataccttacttgcattttatgggcaatataaattattttaaatgaatttgtgatgcttaactttaaattaaaaatgcagatgacctgggttggcattaggcatctgtattacagatgtgtaatttacatatgacagttagagagccagtcatctcaataaaccatattttggtctgtgccaaatttcagtctatggtgtaatagagagtcctccctagaataaataccctcatttccctttagtcagcaagttatcttttctagctagcttattgagcaatagatcaaaacacctatgggtaagaattgccaaattagatctacaggaagattaagtcctgatgtaagacttccctgatattttaataacgatagcagattctatgtgacaggcaccattatcactgtctttcatttattaattcatttaatcttcccaactataagatactctttctcccattttgcaatggggaaactgaggcacagaattaagtatcttgcccacgatcacacagacagataattacttccttgacttctgggagaaaccagagccaggtgctgctttatagcattgtggctatttctggggtttgtacagcgtagctgccttatgtgtgagcatcttctaatgtgatgccttcgtgcttaatgaactttaccgccttggataatagctaccatttgagtaatctctgcgagtcacatctaagaatttgtttgcattattttatgtacttctcaaaagccataagtgctattgtctcttttctaagtcttcctatcttttaactataaacctaaggaacttgcccaagtcctaccactaacctgggagtgaatctgcctgtgtctagagttggcattcttcctcccacaccatgctgctttgtatagattgatctaatgtgaatatatatgataatgaatttattctaatttgtttcagtgaaaacccaaaagaattataacaaaattatcttcagttcaaataatttccatggttataactttaaaataagatcttctgtattctttataactttaagataagatcttctatattcttttaagaatattggtactcttcaggtaatcaaatgggtcatataccttattttgattgtattttaggtccatggtgtggtgaatgatacaatagcatttgtgaagaacatcattaccacagaactgaacagcgcaacagataatcctgtatcttttgattgtatactaacctgcaattatagaattgcagctggtaattttaggatctactgaaacaatcattttgtgtagcttctcagttccattgctccatttcacagagtaaatgaatcagtacctgaggctcagaggttaactgactttctccaggtcagagctggtaagtggcagaggtgacctgtgaacttactctcaggttccaagtctaacatgctgtactttcaaaatggacctattcaagatttaagaagatatttccagcattggctgacatgtttcctgaagacactggccctaaaacccactgccaaggttgttggtggaagggtgaagacgtgggtcacaaatgtgattgcagaaatgctgaacaatttatgcaggcctctctgctttgagtgagagcagtttgcattatgcagaggtcagccttttaacacttaccaaagtattgattaaagagttatggcttcctcatcctgaggtttttggcatttttttttttgtttatttttagacggagtctcactctgtctcctaggctggagtgcagtggtgccatctcggctctctgcaacctctgcctcacgggttcaagcgattctcctgcctcagcctcctgagtagctggaattacaggcatgtgccagcaggcctggctaatatttgtatttttagtagagacagggttttgctgtgttggccaggctggtctcaaactcctgacctcaagtgatccacctgcctcggcctcccaaagtgccgggattacaggtgtgagcgactacacccagcccccatcatgagtttttatttgtcatgcgcactttgctcaaattagtgcttttgcgtttgttcaaagaaatagtaaaactgattccatttctcaaggatggaggtcctctcttaatggtgtggcttatctcagagccatctggtttttctctggaatgttccatgatggctcagttccccagatgttgaggtgatcagtcaggagtcacactggcacagttttcatggagatagcactgcctctgaagataatcaaacgcctttggaatctatttttcctattccacatctgcctcagttctgaatcctagagaaagtgctgagtaagcagccccaaaagcaggtcacaggctgagttagaagatggttatgtgatagctccataaggtttcccatgagatggcttttgtatgtatgtgttttttgacaagcttacttcagaatgtgtgttttatgttctctcacattgttttacactgaagtcaaacccacactaaataagcctcagaaattgtgggtctctccattcacctcaataccaccccatcccccagatggaatagtagtttttatgaactctatgtagtcttatctcacgtaccagtcttttcactgtagggaagaggtatctattctacatttacatcattgcttttaaagggtatctcatagtttttagaaattaccattcaattactgaatatggggcaggggataattaattcctcactaaattcttctgctaccaattgcctcagcaagaatttcagccaaaagtcttttatttttttcaattaaagattgctaattattgataaatttccaataggctaatatttgggagttctcttgcaatactgctgttacctgaaagaatatgtgtctcaattttctgttgcagatacggagccccatcacacatgctggatattatgggagttaaattctaattgttttaatctaatagccaacaaacaaatttggaacaactagaggacaataaaaacatgtctaagttctaaattgatgcagaattccattgctatagggattcagagaatggagaagaggccaagaccaaggagctagacccaatgggctcacagttacacatgcagaacagagtaatccacacttgagtaggttaatgtaacacatacatctctaactctttcaaagttagaaactttcttgcacacagcgagttctcagttaataggtaatgaatattcttgagcccaagaggcaaagattagctctttagaaccaggaagaaggatctgttactgccttttcctgggtgcttgctggtttaagtaaagtgcatgatggaatagaattatctgaggctgggtttggtggctcacacctgtaatcccagcactttgggaggccaaggtgggctgatcacttgaggccaagagtccaagaccagcctggccaacatggcaaaaccctgtctctactaaaaatacaaaatttaactgggtgtgatggtgcatgcctgtaatcccagctactcaggaggctgaggcacaagaaatcacttgaacccgggaggcagaggtcgcagtgagccgagatggcgcccctgcactccagcctgggtgacagaatgatacagaaaataaaaaaagaattacctcagagtaataatattcagcaaatttaaacttgccacctacttaaaatggattcatgaggacatattggccaccatctgctgttgaacgctgacaccagattactgagtaaagaatttgaaaattttctcaaaattgattgttccagacattttggtagttgctcatcagcagagtttcccattcaacagattcttctatggaacacaaatttgcagtgcccattgaagaaacagaagttgctttcaaacagatgtgttggtctctgtttttagtttcaggctataaaccttttgagggcaggtactaaccaccaggttagtacagttatggtgctttagaatctaatctcaagagaaaacatcatttcaaggttcatgttttttcagcctccaaattgggtgtacatgatccacctttaaggctttttgttttttgcttttgtgcccctttatatctcttgcaggaagacttgtctctttccctccaccccacatttgtacacagactgccacctccacgttaaaaaagaaggcaggaaggggttgtacttgaagtgaccagcaaacattatcttcaagccttaacctcttttgaaagatggtctttgccaataggggagagacagtttctggaggaaacttccatggtgaatacccagccaaagtaagctttttaaaactgctcctgacccagaaggcacatttcaatataggctgactaaatggagaccctctttcaggccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttggtaaggacactgcccctttcccagaacacacgcccgtccatctaagggtctttccatttcacctagaagtatcaaatgtggtctgtgagtgagatctaacttttcaccacttttatctgcatcctagttctctcagtgcaaaggtaacaggttaactctaatatttttcagaaaaaaatagtttaaaaacttcgatactgttatgctgaaatataataatacaataataaggctgtaatggacaatttctctgtggaaagcaatttgacattgcatatgaacttcatctctatagcttttttccaaatctccatttgaagactattggaaaaagactaagatggttatagttaatggtgaaacttgaaccaacctataattaaactttgaacttttaaacctataattaaactttggattcatttcactagatataacaaccactaaaaacataaagatgttattgcaacattaaaaaaatgcttataagaatcaggataccctctgtgaagattgagcttcaatatagtttatttctgactcaagattgtaagagacagattaacacatcagaacagtgcctggcactaagtaagtactaagggcctcatttatgttcattcacttaattgcacaattgtcacctgaggtaagtgctattattgttatctatagatgaatatctgaagatctgagagttaagtgacttgcccaatattgcacagctagcaagatgtaaagcagaattcaaaccctgaattttggcatgcaaaaatgtgcttttacttcaattaatgaggatctcattttaatcaatacttaatcctaaaatagggttgtcaaatttaacaaaaacacaggatgactagttaaatttgaattatttgggacatacttgtactacaaaaattatttttttaatctaaaatcaaaatttaactgggcatcctttactttatctaacaaccctaccctaaaaggcaggatagcatgagttattacatgcgtagatgctagatcagaagcccagctctgccatttactagctctgtgagcctctttatcctgttgatcctcaatttacacctgcataaaatggagatcagtttacctaagggggttgtttggaggattaaatgaattgatgtatataaagcctggcacatcataagcactaaaaaaatactagttgctattaatattattcccaacgaacacaaataaccaactaagagccactgaattcacctcctctgaccggtatcttctatttgcttacttgcttattaataaaatacccctttaacaggtagaaatttctcacaataacgtagtagtgggggagtagtcattgatttctagagaacaggatggacattgggaagcctcatttgaatatgttttagtttcccaagctgcctattgaggttgtttggcttgctttcattgtagtaaaattagtgtttttctttttttggcccttgtaagttatctttccgaacaagccttaccacagtttacttatctgtatagtccacagtttagggtccaaggacttgggacttacagtgctgaaacctggaaagtccttggccaaccaacatgaccttgacaaagtagcatgaccctagtttaaagcgagatcatcttaggatgcgatcccatgcagcatagaaaacaactgaagcctcacccatgaccactcttaaagccagagaaagaggagaagagactttgagtctgacctttcctgacttctaagctagggtgagggaacttcataacctaagtttttgctacttatagtgtagtcctcagaccaacaatatggccatcacctgagagctcgttagaacagcagaatttcacgccccacccaaagctcagtatatttgttataacaaggtccctgagtgattcatgtgcactggaaagcttgagagtcccatgtcttggtcctccctagaattcccccacctcccagatatttctggcagtcatcgagcccctgtttacatccatttatgtcatccttgaacactctgaagtgttaaatcttcttgcccaagatttatgggtccagcctcagtccccaaatgccacatcggaattctggtgacacaaccaaattgacgtcaagtacctgaaaatggccatcatgtctctttctcccctagtctccctggcatgctatatgcttcccaggttgtcatcttggccttgttatattagaattcccagttctcccatctccgtcttacctcagaactcactattgctttagagtcctcacttggggaaaaaaaatgacctgcttccttttcccaaagcagctttctaccatcaggtgcttggataatgtccaagatcttctggagagtgtatcccatgctgtggagcactctgtggaagccacgggtcctttagacagctcatcctgtgaggagcacttcttaactggcactggtctcttgcagtttctgagaacaaggctctgtgccatccctcgtctgttgactccctctccaccagcgcagccacggaggaccacgtctccatgggaggatgggcagcaaggaaagccctcagggtcatcgagcatgtggagcaaggtaatgctgatgagttcggggtggcgggcctgcctgatagaccactgtgcccgtggttctcaagtgggatctcccaccagcaacatcagcatcacctggaaacttgttaggaaggcagactctcaggcctccttgcaggctggctaattcagacactccaggaggagagcggagcaatctgtgtttaacgaaccctccaggtgattctgatcttcactaaagattaagaaccaaggaaccatgcagaactaagctggtcctttggctggctttttttttctctttttttttttttttttttgaggcaggatctctatctgccacccaggctggagtgcagtgttgcaacaatagctcactgcagccttgaactcccaggctcaagtgatcctccttccttagtctcctaagtaactgacacctcaggcaagcaccaccacacctgggtaatttttaaattttttgtaaagatggggtcttgctgtgttgccttggctggtctcaaactcttggcctcaagcgatcctcccacctcacccccaaagtggttttttttttaatgagacttttaaaacatattcaaagtggagagaagagtaccatgaactctcacaccccaccttgattcaacagcacattcaatttatgaagtacactatgaactctagtgagtgcagacaggtcacagtgggagaggatgaagttgtggaattcacggtggacctggaagcaatttctcatcttctagtccagtatccccaccccgcaattcatatggaattggaaaccacgagatgtaaagcatcttgatgaaatgcatacaattatttaatagaactggggctcaagatgaggatcagctcttctttctgacttccttccctacccacatgacccaaacttggccaacaaacctttgttagaggatgtgaatgggggacatggcagattttgctagtgcagatgaatgggcagcttcctgggtaggtgtgcaggtctggtaattattcaggtaaatgtctcaattctccccttctcagcattaaagatatagtcaaggggttagaaaacaggttaaaatggcaactaaataccaaagcaacttcattctaccaatagacagagaacaaaccatctgttagttgggtggtgatgggtgtcaactttgggaaatccacattccttaagcatgaagtcagactttcataagctaccctcttggaatttcttgtttcagtgctggccatcgagctccttgcagcctgccagggcatagagtttctacgtcccctgaaaacaaccactccgctggagaaggtctatgacctggtgcgctctgttgtaaggtaagatcaaacaggtctctgaaaattacatcagttcagcactgtgaaccctggcagggtttcatgtttggctgatgggcggatgtgctctccctgtaggccctggataaaagatcgcttcatggccccggacatcgaggcagcccacaggctgctcctggagcagaaggcaagctggctccttggcttgtttcttaatttttaattgttacaggaaagcaaagtcggggatggagaaacattgccttattctattgttttctggccattccttgggaatgagcaaaaaggtatttgagcaacaacttgcagcaccactaagaagccatataataaacactaagtgcaccagggtgcagccaacggttgaatatctaaggattagaaacagaaatggattttaaatggagttttgggacaatattcatggggaaaaaaataagatgtcaggttcattgatgcctccccacccctgcaagatgtcctctttgattcaaaagtgcgttcccttctgggtgatgctatcacaactaccaccactgggtgcaatggctgcgtagacctggagagaaaaccaaggcccgggtgtttacttcagaagactttcggtctgtctagtgctctggtgagatgcgggtttttctttcttgcatatttccactggttacattgaccgtatactcagctgccaggtacagatgcccagcatgctggcttatcttttaaatatcgtttttgaagtgctttaaaccttttgaataacttgtgggtgttatgttccctagaatcgcttgatttccttaaaacagttgctctgctctggggcagtgatttttccctgtgattttcacacccattatttctaagcatgccctgcactctggatggggaagctgcttgcctgctgccaactttggtgctaggagggaggtatacgagaggttagcctttaagagtattcatttgctaaggtcataaaaaataataggaaaggctccttggctgtcagtggcaatcagttaaaaagcagatataaaccaaaaacagggccttaatttcattcaatttaaaaaaaaatttttttagagatgcgggtcttgctatgttgccaaggctggactcaaactcctgggctcaagtgatcctcccactttggcttcttgagtagctgggactacaggcacatgccaccatgcccgtccagggccttactttgagatgatgttatgcttaagaaaaaaaaaaaaaaagaaaaaagaggttggtgggtgggaggcaaaagctaaccaaggatctgaggcagtgcctctccaacattaaagtggctatgaatctgctgggcatcttgttaaaatgcaggtgtggattcagtaggtctgggtgaaggccaagattctgacttgtcccaggtgatgctgatactgcaggggcacaggccacgctttgagtagcagggtcaaaggaacagagcacctgtggcatggaaataaggcaatagggaaatacaattcactgccttttaccatttaaggcttgctttagaaaacacacaggtccaggccatgtgtggtggctcacgcctgtaatcccagcactttgggaagctgtggtgggtggatcacttgaggccagaaattcgagaccagcctggccaacatggtgaaaccccatctctgctaaaaatacaaaaattagctgggcctggtggcatgcacctgtaatcccagctactcaggaggctgaggcacaagaattgcttgaacctggaaggtggaggttgcagtgagctgagatcatgctactgcactccagcctgggcgacagagtgagactctgtctcacaaaaaaagaaaaaaaacaaaaaaacaagaaaaaaaccacataggtactgttaagaccctggagcaactatccttcactctatccacactgaattctctgggttttgttatttgcttgcttgttttgcttaggaaaagtatgcagcttaaaatctttatgatgactttagaaagttttggactttgtatatttacagtctttaatgcaaccaaagactaattgacacaggtcatgtgtcaaatggcttaaaataaaagcaagcaaacagcagctgtgtctggtcatgtatagagtatgttttgacttcggatgaagttgatgtttaatagcatgtggaaacaatgtcgtcaatatttcaactttgttttgtccttgcttttcaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataagtcctattgataaaggtcagatggagccctagagtgtattactgcatctatttttttccccgagaagataaaggaccttcagggatggcttaagtgtatctgtccagatgaaggatgggtcacatgacctcttggcttcccaagtctaagctctgtgactttgcaccagtgtgtgcatatatgtgcaaggcccttcaagtggtctgaaaccgtggctctaaaaaccacagctggtggagaggaggacagacacacttgccaccttgcctacctaattgccatctaaaatgggccgaacagtggatttcacaatagagttttcaccctttagatttacaacctgtcaggtggaaactgaagtgaaaactgctgcacacagcaattcagggagcaaaaaatgtgctgaggagactgtttacctaaaggttgttcttggtgctattccttgtcaaaatgtgaacacacacaaatgaggtttgtgcattgtcatccgtgggctgccattgagccagtaacccccagtggtctcatggtgctcttcgctccagtttggggaatgctggattctttcagcccctgcagccctccaggtcaaaatgacactttgtcactgagttttctacacagctctattagtaactgacagcacacgccttcaagggaacttcaagggaaacatggaataaactaagtctcaattgcc

SEQ ID NO: 4 - RNA, 3892 nt, Homo sapiens
  source 1..3892: mol_type = genomic RNA
agataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagttgtttacggtattactacaggttttgggaaatttgccagaactgtaattcctatcaataagctacaggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataagtcctattgataaaggtcagatggagccctagagtgtattactgcatctatttttttccccgagaagataaaggaccttcagggatggcttaagtgtatctgtccagatgaaggatgggtcacatgacctcttggcttcccaagtctaagctctgtgactttgcaccagtgtgtgcatatatgtgcaaggcccttcaagtggtctgaaaccgtggctctaaaaaccacagctggtggagaggaggacagacacacttgccaccttgcctacctaattgccatctaaaatgggccgaacagtggatttcacaatagagttttcaccctttagatttacaacctgtcaggtggaaactgaagtgaaaactgctgcacacagcaattcagggagcaaaaaatgtgctgaggagactgtttacctaaaggttgttcttggtgctattccttgtcaaaatgtgaacacacacaaatgaggtttgtgcattgtcatccgtgggctgccattgagccagtaacccccagtggtctcatggtgctcttcgctccagtttggggaatgctggattctttcagcccctgcagccctccaggtcaaaatgacactttgtcactgagttttctacacagctctattagtaactgacagcacacgccttcaagggaacttcaagggaaacatggaataaactaagtctcaattgcc

SEQ ID NO: 5 - RNA, 3846 nt, Homo sapiens
  source 1..3846: mol_type = genomic RNA
gttttacagttgagtaatctcagataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataagtcctattgataaaggtcagatggagccctagagtgtattactgcatctatttttttccccgagaagataaaggaccttcagggatggcttaagtgtatctgtccagatgaaggatgggtcacatgacctcttggcttcccaagtctaagctctgtgactttgcaccagtgtgtgcatatatgtgcaaggcccttcaagtggtctgaaaccgtggctctaaaaaccacagctggtggagaggaggacagacacacttgccaccttgcctacctaattgccatctaaaatgggccgaacagtggatttcacaatagagttttcaccctttagatttacaacctgtcaggtggaaactgaagtgaaaactgctgcacacagcaattcagggagcaaaaaatgtgctgaggagactgtttacctaaaggttgttcttggtgctattccttgtcaaaatgtgaacacacacaaatgaggtttgtgcattgtcatccgtgggctgccattgagccagtaacccccagtggtctcatggtgctcttcgctccagtttggggaatgctggattctttcagcccctgcagccctccaggtcaaaatgacactttgtcactgagttttctacacagctctattagtaactgacagcacacgccttcaagggaacttcaagggaaacatggaataaactaagtctcaattgcc

SEQ ID NO: 6 - RNA, 2637 nt, Homo sapiens
  source 1..2637: mol_type = genomic RNA
agataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagttgtttacggtattactacaggttttgggaaatttgccagaactgtaattcctatcaataagctacaggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttt

SEQ ID NO: 7 - RNA, 3822 nt, Homo sapiens
  source 1..3822: mol_type = genomic RNA
agataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagttgtttacggtattactacaggttttgggaaatttgccagaactgtaattcctatcaataagctacaggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataagtcctattgataaaggtcagatggagccctagagtgtattactgcatctatttttttccccgagaagataaaggaccttcagggatggcttaagtgtatctgtccagatgaaggatgggtcacatgacctcttggcttcccaagtctaagctctgtgactttgcaccagtgtgtgcatatatgtgcaaggcccttcaagtggtctgaaaccgtggctctaaaaaccacagctggtggagaggaggacagacacacttgccaccttgcctacctaattgccatctaaaatgggccgaacagtggatttcacaatagagttttcaccctttagatttacaacctgtcaggtggaaactgaagtgaaaactgctgcacacagcaattcagggagcaaaaaatgtgctgaggagactgtttacctaaaggttgttcttggtgctattccttgtcaaaatgtgaacacacacaaatgaggtttgtgcattgtcatccgtgggctgccattgagccagtaacccccagtggtctcatggtgctcttcgctccagtttggggaatgctggattctttcagcccctgcagccctccaggtcaaaatgacactttgtcactgagttttctacacagctctattagtaactgacagcacacgccttcaagggaacttcaagggaaacatggaataaactaagtctcaattgcc

SEQ ID NO: 8 - RNA, 3825 nt, Homo sapiens
  source 1..3825: mol_type = genomic RNA
agataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataagtcctattgataaaggtcagatggagccctagagtgtattactgcatctatttttttccccgagaagataaaggaccttcagggatggcttaagtgtatctgtccagatgaaggatgggtcacatgacctcttggcttcccaagtctaagctctgtgactttgcaccagtgtgtgcatatatgtgcaaggcccttcaagtggtctgaaaccgtggctctaaaaaccacagctggtggagaggaggacagacacacttgccaccttgcctacctaattgccatctaaaatgggccgaacagtggatttcacaatagagttttcaccctttagatttacaacctgtcaggtggaaactgaagtgaaaactgctgcacacagcaattcagggagcaaaaaatgtgctgaggagactgtttacctaaaggttgttcttggtgctattccttgtcaaaatgtgaacacacacaaatgaggtttgtgcattgtcatccgtgggctgccattgagccagtaacccccagtggtctcatggtgctcttcgctccagtttggggaatgctggattctttcagcccctgcagccctccaggtcaaaatgacactttgtcactgagttttctacacagctctattagtaactgacagcacacgccttcaagggaacttcaagggaaacatggaataaactaagtctcaattgcc

SEQ ID NO: 9 - RNA, 3085 nt, Homo sapiens
  source 1..3085: mol_type = genomic RNA
agcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagttgtttacggtattactacaggttttgggaaatttgccagaactgtaattcctatcaataagctacaggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataag

SEQ ID NO: 10 - RNA, 2804 nt, Homo sapiens
  source 1..2804: mol_type = genomic RNA
agacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacaatttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcggccacggaggaccacgtctccatgggaggatgggcagcaaggaaagccctcagggtcatcgagcatatggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgatt

SEQ ID NO: 11 - RNA, 1474 nt, Homo sapiens
  source 1..1474: mol_type = genomic RNA
gagtaatctcagataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcgcctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtataattcagttcttctaaagcctacgttggttaggccgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagacgaattcaaatttggaaacagattgatagcaattttttctaaaaacattagacttttgttaaccttttttttttttttttttttaaatttgcttc

SEQ ID NO: 12 - RNA, 2271 nt, Homo sapiens
  source 1..2271: mol_type = genomic RNA
gccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccgctctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacaatttctggaggaaacttccatggccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcag

SEQ ID NO: 13 - RNA, 2355 nt, Homo sapiens
  source 1..2355: mol_type = genomic RNA
gccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagttgtttacggtattactacaggttttgggaaatttgccagaactgtaattcctatcaataagctacaggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacaatttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcag

SEQ ID NO: 14 - RNA, 3913 nt, Homo sapiens
  source 1..3913: mol_type = genomic RNA
gttttacagttgagtaatctcagataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagttgtttacggtattactacaggttttgggaaatttgccagaactgtaattcctatcaataagctacaggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataagtcctattgataaaggtcagatggagccctagagtgtattactgcatctatttttttccccgagaagataaaggaccttcagggatggcttaagtgtatctgtccagatgaaggatgggtcacatgacctcttggcttcccaagtctaagctctgtgactttgcaccagtgtgtgcatatatgtgcaaggcccttcaagtggtctgaaaccgtggctctaaaaaccacagctggtggagaggaggacagacacacttgccaccttgcctacctaattgccatctaaaatgggccgaacagtggatttcacaatagagttttcaccctttagatttacaacctgtcaggtggaaactgaagtgaaaactgctgcacacagcaattcagggagcaaaaaatgtgctgaggagactgtttacctaaaggttgttcttggtgctattccttgtcaaaatgtgaacacacacaaatgaggtttgtgcattgtcatccgtgggctgccattgagccagtaacccccagtggtctcatggtgctcttcgctccagtttggggaatgctggattctttcagcccctgcagccctccaggtcaaaatgacactttgtcactgagttttctacacagctctattagtaactgacagcacacgccttcaagggaacttcaagggaaacatggaataaactaagtctcaattgcc

SEQ ID NO: 15 - DNA, 3892 bp, Homo sapiens
  source 1..3892: mol_type = genomic DNA
agataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagttgtttacggtattactacaggttttgggaaatttgccagaactgtaattcctatcaataagctacaggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataagtcctattgataaaggtcagatggagccctagagtgtattactgcatctatttttttccccgagaagataaaggaccttcagggatggcttaagtgtatctgtccagatgaaggatgggtcacatgacctcttggcttcccaagtctaagctctgtgactttgcaccagtgtgtgcatatatgtgcaaggcccttcaagtggtctgaaaccgtggctctaaaaaccacagctggtggagaggaggacagacacacttgccaccttgcctacctaattgccatctaaaatgggccgaacagtggatttcacaatagagttttcaccctttagatttacaacctgtcaggtggaaactgaagtgaaaactgctgcacacagcaattcagggagcaaaaaatgtgctgaggagactgtttacctaaaggttgttcttggtgctattccttgtcaaaatgtgaacacacacaaatgaggtttgtgcattgtcatccgtgggctgccattgagccagtaacccccagtggtctcatggtgctcttcgctccagtttggggaatgctggattctttcagcccctgcagccctccaggtcaaaatgacactttgtcactgagttttctacacagctctattagtaactgacagcacacgccttcaagggaacttcaagggaaacatggaataaactaagtctcaattgcc

SEQ ID NO: 16 - DNA, 3846 bp, Homo sapiens
  source 1..3846: mol_type = genomic DNA
gttttacagttgagtaatctcagataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataagtcctattgataaaggtcagatggagccctagagtgtattactgcatctatttttttccccgagaagataaaggaccttcagggatggcttaagtgtatctgtccagatgaaggatgggtcacatgacctcttggcttcccaagtctaagctctgtgactttgcaccagtgtgtgcatatatgtgcaaggcccttcaagtggtctgaaaccgtggctctaaaaaccacagctggtggagaggaggacagacacacttgccaccttgcctacctaattgccatctaaaatgggccgaacagtggatttcacaatagagttttcaccctttagatttacaacctgtcaggtggaaactgaagtgaaaactgctgcacacagcaattcagggagcaaaaaatgtgctgaggagactgtttacctaaaggttgttcttggtgctattccttgtcaaaatgtgaacacacacaaatgaggtttgtgcattgtcatccgtgggctgccattgagccagtaacccccagtggtctcatggtgctcttcgctccagtttggggaatgctggattctttcagcccctgcagccctccaggtcaaaatgacactttgtcactgagttttctacacagctctattagtaactgacagcacacgccttcaagggaacttcaagggaaacatggaataaactaagtctcaattgcc

SEQ ID NO: 17 - DNA, 2637 bp, Homo sapiens
  source 1..2637: mol_type = genomic DNA
agataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagttgtttacggtattactacaggttttgggaaatttgccagaactgtaattcctatcaataagctacaggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttt

SEQ ID NO: 18 - DNA, 3822 bp, Homo sapiens
  source 1..3822: mol_type = genomic DNA
agataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagttgtttacggtattactacaggttttgggaaatttgccagaactgtaattcctatcaataagctacaggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataagtcctattgataaaggtcagatggagccctagagtgtattactgcatctatttttttccccgagaagataaaggaccttcagggatggcttaagtgtatctgtccagatgaaggatgggtcacatgacctcttggcttcccaagtctaagctctgtgactttgcaccagtgtgtgcatatatgtgcaaggcccttcaagtggtctgaaaccgtggctctaaaaaccacagctggtggagaggaggacagacacacttgccaccttgcctacctaattgccatctaaaatgggccgaacagtggatttcacaatagagttttcaccctttagatttacaacctgtcaggtggaaactgaagtgaaaactgctgcacacagcaattcagggagcaaaaaatgtgctgaggagactgtttacctaaaggttgttcttggtgctattccttgtcaaaatgtgaacacacacaaatgaggtttgtgcattgtcatccgtgggctgccattgagccagtaacccccagtggtctcatggtgctcttcgctccagtttggggaatgctggattctttcagcccctgcagccctccaggtcaaaatgacactttgtcactgagttttctacacagctctattagtaactgacagcacacgccttcaagggaacttcaagggaaacatggaataaactaagtctcaattgcc

SEQ ID NO: 19 - DNA, 3825 bp, Homo sapiens
  source 1..3825: mol_type = genomic DNA
agataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataagtcctattgataaaggtcagatggagccctagagtgtattactgcatctatttttttccccgagaagataaaggaccttcagggatggcttaagtgtatctgtccagatgaaggatgggtcacatgacctcttggcttcccaagtctaagctctgtgactttgcaccagtgtgtgcatatatgtgcaaggcccttcaagtggtctgaaaccgtggctctaaaaaccacagctggtggagaggaggacagacacacttgccaccttgcctacctaattgccatctaaaatgggccgaacagtggatttcacaatagagttttcaccctttagatttacaacctgtcaggtggaaactgaagtgaaaactgctgcacacagcaattcagggagcaaaaaatgtgctgaggagactgtttacctaaaggttgttcttggtgctattccttgtcaaaatgtgaacacacacaaatgaggtttgtgcattgtcatccgtgggctgccattgagccagtaacccccagtggtctcatggtgctcttcgctccagtttggggaatgctggattctttcagcccctgcagccctccaggtcaaaatgacactttgtcactgagttttctacacagctctattagtaactgacagcacacgccttcaagggaacttcaagggaaacatggaataaactaagtctcaattgcc

SEQ ID NO: 20 - DNA, 3085 bp, Homo sapiens
  source 1..3085: mol_type = genomic DNA
agcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagttgtttacggtattactacaggttttgggaaatttgccagaactgtaattcctatcaataagctacaggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataag

SEQ ID NO: 21 - DNA, 2804 bp, Homo sapiens
  source 1..2804: mol_type = genomic DNA
agacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacaatttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcggccacggaggaccacgtctccatgggaggatgggcagcaaggaaagccctcagggtcatcgagcatatggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgatt

SEQ ID NO: 22 - DNA, 1474 bp, Homo sapiens
  source 1..1474: mol_type = genomic DNA
gagtaatctcagataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcgcctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtataattcagttcttctaaagcctacgttggttaggccgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagacgaattcaaatttggaaacagattgatagcaattttttctaaaaacattagacttttgttaaccttttttttttttttttttttaaatttgcttc

SEQ ID NO: 23 - DNA, 2271 bp, Homo sapiens
  source 1..2271: mol_type = genomic DNA
gccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccgctctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacaatttctggaggaaacttccatggccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcag

SEQ ID NO: 24 - DNA, 2355 bp, Homo sapiens
  source 1..2355: mol_type = genomic DNA
gccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagttgtttacggtattactacaggttttgggaaatttgccagaactgtaattcctatcaataagctacaggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacaatttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcag

SEQ ID NO: 25 - DNA, 3913 bp, Homo sapiens
  source 1..3913: mol_type = genomic DNA
gttttacagttgagtaatctcagataaagacaggaacatggtgtttataagctgcccttaatggcagagaacacagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagttgtttacggtattactacaggttttgggaaatttgccagaactgtaattcctatcaataagctacaggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataagtcctattgataaaggtcagatggagccctagagtgtattactgcatctatttttttccccgagaagataaaggaccttcagggatggcttaagtgtatctgtccagatgaaggatgggtcacatgacctcttggcttcccaagtctaagctctgtgactttgcaccagtgtgtgcatatatgtgcaaggcccttcaagtggtctgaaaccgtggctctaaaaaccacagctggtggagaggaggacagacacacttgccaccttgcctacctaattgccatctaaaatgggccgaacagtggatttcacaatagagttttcaccctttagatttacaacctgtcaggtggaaactgaagtgaaaactgctgcacacagcaattcagggagcaaaaaatgtgctgaggagactgtttacctaaaggttgttcttggtgctattccttgtcaaaatgtgaacacacacaaatgaggtttgtgcattgtcatccgtgggctgccattgagccagtaacccccagtggtctcatggtgctcttcgctccagtttggggaatgctggattctttcagcccctgcagccctccaggtcaaaatgacactttgtcactgagttttctacacagctctattagtaactgacagcacacgccttcaagggaacttcaagggaaacatggaataaactaagtctcaattgcc

SEQ ID NO: 26 - protein, 657 aa, Homo sapiens
MPRYTVHVRGEWLAVPCQDAQLTVGWLGREAVRRYIKNKPDNGGFTSVDDAHFLVRRCKGLGLLDNEDRLEVALENNEFVEVVIEGDAMSPDFIPSQPEGVYLYSKYREPEKYIELDGDRLTTEDLVNLGKGRYKIKLTPTAEKRVQKSREVIDSIIKEKTVVYGITTGFGKFARTVIPINKLQELQVNLVRSHSSGVGKPLSPERCRMLLALRINVLAKGYSGISLETLKQVIEMFNASCLPYVPEKGTVGASGDLAPLSHLALGLVGEGKMWSPKSGWADAKYVLEAHGLKPVILKPKEGLALINGTQMITSLGCEAVERASAIARQADIVAALTLEVLKGTTKAFDTDIHALRPHRGQIEVAFRFRSLLDSDHHPSEIAESHRFCDRVQDAYTLRCCPQVHGVVNDTIAFVKNIITTELNSATDNPMVFANRGETVSGGNFHGEYPAKALDYLAIGIHELAAISERRIERLCNPSLSELPAFLVAEGGLNSGFMIAHCTAAALVSENKALCHPSSVDSLSTSAATEDHVSMGGWAARKALRVIEHVEQVLAIELLAACQGIEFLRPLKTTTPLEKVYDLVRSVVRPWIKDRFMAPDIEAAHRLLLEQKVWEVAAPYIEKYRMEHIPESRPLSPTAFSLQFLHKKSTKIPESEDL

SEQ ID NO: 27 - protein, 449 aa, Homo sapiens
MLLALRINVLAKGYSGISLETLKQVIEMFNASCLPYVPEKGTVGASGDLAPLSHLALGLVGEGKMWSPKSGWADAKYVLEAHGLKPVILKPKEGLALINGTQMITSLGCEAVERASAIARQADIVAALTLEVLKGTTKAFDTDIHALRPHRGQIEVAFRFRSLLDSDHHPSEIAESHRFCDRVQDAYTLRCCPQVHGVVNDTIAFVKNIITTELNSATDNPMVFANRGETVSGGNFHGEYPAKALDYLAIGIHELAAISERRIERLCNPSLSELPAFLVAEGGLNSGFMIAHCTAAALVSENKALCHPSSVDSLSTSAATEDHVSMGGWAARKALRVIEHVEQVLAIELLAACQGIEFLRPLKTTTPLEKVYDLVRSVVRPWIKDRFMAPDIEAAHRLLLEQKVWEVAAPYIEKYRMEHIPESRPLSPTAFSLQFLHKKSTKIPESEDL

SEQ ID NO: 28 - protein, 591 aa, Homo sapiens
MPRYTVHVRGEWLAVPCQDAQLTVGWLGREAVRRYIKNKPDNGGFTSVDDAHFLVRRCKGLGLLDNEDRLEVALENNEFVEVVIEGDAMSPDFIPSQPEGVYLYSKYREPEKYIELDGDRLTTEDLVNLGKGRYKIKLTPTAEKRVQKSREVIDSIIKEKTVVYGITTGFGKFARTVIPINKLQELQVNLVRSHSSGVGKPLSPERCRMLLALRINVLAKGYSGISLETLKQVIEMFNASCLPYVPEKGTVGASGDLAPLSHLALGLVGEGKMWSPKSGWADAKYVLEAHGLKPVILKPKEGLALINGTQMITSLGCEAVERASAIARQADIVAALTLEVLKGTTKAFDTDIHALRPHRGQIEVAFRFRSLLDSDHHPSEIAESHRFCDRVQDAYTLRCCPQVHGVVNDTIAFVKNIITTELNSATDNPMVFANRGETVSGGNFHGEYPAKALDYLAIGIHELAAISERRIERLCNPSLSELPAFLVAEGGLNSGFMIAHCTAAALVSENKALCHPSSVDSLSTSAATEDHVSMGGWAARKALRVIEHVEQVLAIELLAACQGIEFLRPLKTTTPLEKVYDLVRSVVRFGK

SEQ ID NO: 29 - protein, 219 aa, Homo sapiens [sequence omitted]

SEQ ID NO: 30 - protein, 167 aa, Homo sapiens
MPRYTVHVRGEWLAVPCQDAQLTVGWLGREAVRRYIKNKPDNGGFTSVDDAHFLVRRCKGLGLLDNEDRLEVALENNEFVEVVIEGDAMSPDFIPSQPEGVYLYSKYREPEKYIELDGDRLTTEDLVNLGKGRYKIKLTPTAEKRVQKSREVIDSIIKEKTGSFRST